(19) United States
(12) Patent Application Publication
(10) Pub. No.: US 2015/0241353 A1
(43) Pub. Date: Aug. 27, 2015

(54) METHOD AND SYSTEM FOR OPTICAL ANALYSIS

(71) Applicants: KYUSHU UNIVERSITY, NATIONAL UNIVERSITY CORPORATION, Fukuoka (JP); USHIO DENKI KABUSHIKI KAISHA, Tokyo (JP)

(72) Inventors: Yuji OKI, Fukuoka-shi (JP); Kinichi MORITA, Tokyo (JP)

(73) Assignees: KYUSHU UNIVERSITY, NATIONAL UNIVERSITY CORPORATION, Fukuoka (JP); USHIO DENKI KABUSHIKI KAISHA, Tokyo (JP)

(21) Appl. No.: 14/625,180

(22) Filed: Feb. 18, 2015

(30) Foreign Application Priority Data
Feb. 21, 2014 (JP) 2014-032245

(51) Int. Cl. G01N 21/64; B01L 3/00; B01L 99/00; G01N 33/53

(52) U.S. Cl. CPC G01N 21/6486; G01N 33/5302; B01L 3/502715; B01L 99/00; B01L 2300/027; G01N 2201/08; G01N 2201/061

(57) ABSTRACT

A method for optical analysis, which enables a point of care testing to optically analyze a specimen with a disposal microchip. The method uses a portable terminal device having a processing unit, a light receiving unit, and a display unit for displaying processing results of the processing unit. The microchip has a light introducing portion and a light emitting portion, but does not have a light source. The specimen is held in an optical path extending from the light introducing portion to the light emitting portion. The specimen is irradiated with light for analysis of the specimen. The method includes preparing another optical path for guiding light emitted from the light emitting portion of the microchip to the light receiving portion, and introducing the light into the light introducing portion.

### FIELD OF THE INVENTION

The present invention relates to a method and a system for optical analysis, including spectrophotometric analysis, and a program for use in such method and system. In particular, the present invention relates to a method for optical analysis, which uses a portable terminal device having a processing unit (arithmetic calculation unit), a light receiving unit and a display unit adapted to display processing results of the processing unit, and which causes a microchip to irradiate a test target (specimen) with light for analysis of the test target. The microchip does not have a light source. The microchip has a light inlet portion (introducing portion) and a light exit portion, and is configured to hold the test target in an optical path that communicates from the light inlet portion to the light exit portion.

### DESCRIPTION OF THE RELATED ART

In recent years, a microscale analysis channel or the like is formed on a small substrate made from, for example, silicon, silicone, or glass, by a semiconductor fine processing technology to configure a microchip having the small substrate and the microchannel on the substrate. A microreactor having such microchip is used to isolate, synthesize, extract and analyze a trace amount of sample drug (test drug) is becoming popular.

A reaction analysis system that uses the microreactor is referred to as “micro total analysis system,” “μTAS” or microTAS.” When the μTAS is used, a ratio of a surface area of a test drug (sample drug) to a volume becomes large. Thus, a reaction analysis can be carried out at a high speed and high accuracy. It is also possible to make a compact system and automate the system.

The microchip has a fluid passage, which is referred to as a microchannel, provided in the microchip. A test drug is disposed in a reaction area of the microchannel. The microchip also has other areas having various functions, in which fluid control elements and components (e.g., micropumps, microvalves, micromixers, filters and sensors) are provided. These areas are integrated in the microchip such that the microchip can be used in various applications.

Typically, the microchip includes a pair of microchip substrates bonded to each other, and a fine channel (microchannel) formed on the surface of at least one of the two microchip substrates. The fine channel is, for example, 10 to several hundred micrometers in width and 10 to several hundred micrometers in depth.

The microchip is often used in analysis in the fields of chemistry, biochemistry, pharmacology, medical science, and veterinary science, including gene analysis, a clinical diagnosis and a drug screening. The microchip is also often used when synthesizing chemical substances, or measuring environmental data.

For example, when the microchip is used in medicines or medical devices, the microchip is included in (or used as) a preserving container to preserve a living-thing-derived substance (biochemical substance) such as protein, or a analyzing device for such substance. Specifically, the microchip is used in the measurement that takes advantage of intermolecular interaction such as immune reaction in a clinical test or the like (measuring technology using a SPR (surface plasmon resonance), measuring technology using a QCR (quartz crystal microbalance), or measuring technology using a functional surface from a gold colloidal particle to a ultrafine particle.

The microchips can be fabricated at a relatively low cost. Thus, it is possible to prepare and use the microchips in a large quantity depending upon a required quantity in a chemical analysis. The microchips can be therefore treated as the disposal devices. It is possible to omit the cleaning and maintenance works after the analysis, unlike ordinary analyzing devices. The cleaning and maintenance works are often troublesome.

Various chemical operations such as mixing of solutions, reactions, isolation, separation, refining and detection can take place in the microchip. When the microchip(s) is incorporated in an analyzing device, the analyzing device detects reactions and other phenomena that take place in the microchip(s). For example, when the microchip is used as an SPR (surface plasmon resonance) sensor, the analyzing device may include a light source having a laser unit (or other light emitting element) to emit monochromatic light, and a light receiving unit to receive light from the microchip. The microchip is incorporated in an analyzing device dedicated to a particular use, so as to enable a desired analysis.

On the other hand, many of conventional analyzing devices dedicated to a particular use include large and expensive laser units and/or large and expensive microscopes to carry out desired detection. To deal with such shortcoming, size reduction of the light source and the detecting system, including the detectors, is studied for the analysis-dedicated device.

For example, Patent Literature 1 (Japanese Patent Application Laid-Open Publication No. 2005-535871 or PCT International Publication No. WO 2003/102554) discloses a detection system that is used with a microchip. The detection system uses a laser diode and an integrated type laser-induced fluorescence detecting element.

Non-Patent Literature 1 (will be mentioned below) discloses the integration of OLED (organic light emitting diodes) into a microchip.

FIGS. 22A to 22C of the accompanying drawings schematically illustrate an analyzing process that uses a microchip.

Firstly, as shown in FIG. 22A, a specimen (object to be analyzed) 201 is taken out by a micropipette 203 by a necessary amount for analysis. The specimen 201 is obtained from, for example, a human body, an animal, river or wasted liquid. It should be noted that a pretreatment may be conducted before the specimen 201 is taken out by the micropipette 203 to remove impurities or the like, if necessary. Then, the specimen 201 is dropped into a fluid passage of a microchip 205 from the micropipette 203, as shown in FIG. 22B.

The specimen 201 is received in the microchip 205 and a reaction of the specimen 201 takes place (e.g., a biomolecular reaction between an antigen and an antibody) in the microchip 205. Subsequently, the microchip 205 is loaded in an analyzing device 207, as shown in FIG. 22C. The reaction of the specimen 201 is detected by the analyzing device 207 with light emitted from a light source of the analyzing device 207. The detection results, in the form of detection signals, are processed by a control device 209. The control device 209 processes and analyzes the detection signals. The control device 209 is also used to regulate and control various setting of the analyzing device 207, log-in data and send data. The analysis-dedicated device includes the analyzing device 207 and the control device 209.

In the life science technology of recent years, there is an increasing demand for POCT (point of care testing). In other words, there is an increasing demand for a compact and portable measuring device that performs the testing in a short time and provides evaluation and analysis at a high accuracy at a location where the analysis results are necessary.

### LISTING OF REFERENCES

### Patent Literatures

PATENT LITERATURE 1: Japanese Patent Application Laid-Open Publication No. 2005-535871 (WO 2003/102554)
PATENT LITERATURE 2: Japanese Patent Application Laid-Open Publication No. 2009-84128
PATENT LITERATURE 3: Japanese Patent Application Laid-Open Publication No. 2007-298502
PATENT LITERATURE 4: Japanese Patent Application Laid-Open Publication No. 2009-109232
PATENT LITERATURE 5: Japanese Patent Application Laid-Open Publication No. 2012-76016

### Non-Patent Literatures

NON-PATENT LITERATURE 1: College of Industrial Technology, Nihon University, No. 41 (Heisei 20) Scientific Presentation Poster 5, Applied Molecular Chemistry Section Meeting 5-64, “Developments in Microchip Fluorescence Detecting System Using Organic EL Light Source,” Hizuru Nagajima, et al. URL (searched Dec. 20, 2012): http c.cit.nihon-u.ac./kenkyu/kouennkai/reference/No.41/5_ouka/5-064.pdf>

### SUMMARY OF THE INVENTION

Although the microchip itself is small (compact) and portable, the measuring device is not always small and portable. As described above, the conventional analysis-dedicated device has a large laser (large light source) unit and a large microscope. Usually the conventional analysis-dedicated device is installed in a research institute, and not portable.

The detecting system disclosed in Patent Literature 1, which uses a laser diode and an active element (or elements) of an integrated type laser-induced fluorescence detecting element, is compact and portable, but it is configured and designed for a particular analysis. In order to cope with a variety of analyses, therefore, a large number of detecting systems should be prepared. The laser-induced fluorescence detecting element has an amorphous silicon photodiode, and an optical interference filter integrated and patterned on the amorphous silicon photodiode. The optical interference filter is thick and made from SiO₂/Ta₂O₅. The laser-induced fluorescence detecting element, therefore, has a complicated structure and is expensive.

When the OLEDs (organic light emitting diodes) are integrated in a microchip as disclosed in the Non-Patent Literature 1, the microchip may be integrated with the detection system, and therefore the characteristics of the microchip (i.e., being compact and portable) are maintained. However, because the active element is included, the microchip becomes expensive. In addition, because a battery is integrated to the microchip to feed an energy to the active element, it is difficult to use the microchip as a disposal device when the cost is considered.

An object of the present invention is to provide a method for optical analysis, which enables a point of care testing to optically analyze a specimen using a disposal microchip that has no light source.

According to one aspect of the present invention, there is provided an improved method for optical analysis. The method includes preparing a portable terminal device having an operating and calculating unit, a light receiving unit, and a display unit configured to display processing results of the operating and calculating unit. The method also includes preparing a microchip having a light inlet portion and a light outlet portion, but having no light source. The microchip is configured to hold a specimen (object to be analyzed) in a first optical path extending from the light inlet portion to the light outlet portion. The method also includes preparing a second optical path configured to guide light exiting from the light outlet portion of the microchip to the light receiving unit. The method also includes introducing light into the light inlet portion of the microchip to irradiate the specimen in the first optical path with the light. The method also includes guiding the light, which is emitted from the irradiated specimen, to the light receiving unit through the second optical path. The method also includes analyzing the light, which is received at the light receiving unit, by the operating and calculating unit. This method allows a person, who is not an expert of optical analysis, to conduct a POCT (point of care testing) for analysis of a specimen at a place where a test needs to be conducted (e.g., at a harbor, in a factory, at home, or in a hospital), with a disposal microchip and an ordinary portable terminal device at a low cost in an easy manner. The optical analysis may include spectrophotometric analysis.

In the step of introducing light into the light inlet portion of the microchip, an external light source other than the portable terminal device may be used to introduce the light into the light inlet portion of the microchip. The external light source may emit ultraviolet light or infrared light. The external light source may include an LED. Then, the POCT is carried out with an optimal wavelength for the optical analysis in consideration of given conditions because the ultraviolet light, the infrared light, or the light from the LED may be used in consideration of circumstances. A light condensing device such as an optical fiber may be used with the method. With the light condensing device, it is possible to emit the light having a necessary intensity with a smaller electric power than when a display portion (display unit) of the portable terminal device is used as a light source.

In the step of introducing light into the light inlet portion of the microchip, the portable terminal device may feed electricity to the external light source. Then, the portable terminal device can be used as an electric power source, and it becomes possible to perform the POCT at, for example, an outdoor place where power supply from a commercial power supply system is difficult.

The portable terminal device may further include a control unit configured to control the display unit. During the step of introducing light into the light inlet portion of the microchip, the control unit may control the display unit to reduce or stop light emission from the display unit. Then, the electric power, which would otherwise be spent for the light emission from the display unit, may be supplied to the external light source. The light emission from the display unit may be reduced or stopped because the light emission from the display unit may become noises to the optical analysis. This facilitates the POCT at high precision.

Prior to the step of introducing light into the light inlet portion of the microchip, the method may include the step of determining whether or not a remaining battery energy of the portable terminal device is equal to or greater than a value which is sufficient to feed the electricity to the external light source. Then, it becomes possible to confirm if the remaining amount of the battery is sufficient to supply the electricity to the external light source, before performing the POCT. The external light source emits light to be introduced to the light inlet portion of the microchip.

Light emitted from the external light source may be different from light emitted from the display unit in terms of at least one of wavelength and intensity. The light emitted from the external light source may include pulsed light, coherent light, terahertz light, and/or polarized light. The light emitted from the display unit is usually visible light. When the light emitted from the display unit cannot achieve or is not suitable for the desired optical analysis, then the light emitted from the external light source is used to perform the desired optical analysis. Use of such external light source is also effective when operating an optically driven device (e.g., light-driven pump or the like) used for the optical analysis. Specifically, the optically driven device is irradiated with the light emitted from the external light source, which is more suitable than the light emitted from the display unit. This facilitates an effective activation and manipulation of the optically driven device.

Prior to the step of introducing light into the light inlet portion of the microchip, the method may further include the step of determining whether the light receiving unit functions normally. It becomes possible to confirm, prior to conducting the POCT, whether or not the light receiving unit is ready.

According to another aspect of the present invention, there is provided a system for optical analysis, which includes a portable terminal device and a microchip. The portable terminal device has an operating and calculating unit, a light receiving unit, and a display unit configured to display processing results of the operating and calculating unit. The microchip has a light inlet portion and a light outlet portion such that a specimen is held in a first optical path extending from the light inlet portion to the light outlet portion. The specimen is irradiated with light for analysis of the specimen. The system also includes a second optical path configured to guide light exiting from the light outlet portion of the microchip to the light receiving unit such that the operating and calculating unit of the portable terminal device analyzes the light which is received at the light receiving unit.

The system may further include an external light source configured to receive electricity from the portable terminal device. The external light source may be used to introduce the light into the light inlet portion of the microchip. The external light source may emit ultraviolet light or infrared light. The external light source may include an LED. Then, the POCT is carried out with an optimal wavelength for the optical analysis in consideration of given conditions because the ultraviolet light, the infrared light, or the light from the LED may be used in consideration of circumstances.

The second optical path may have a light condensing portion configured to enhance an intensity of the light directed to the light receiving unit. This makes it possible to send the light to the light receiving unit at a sufficient optical intensity even when the electricity supply is given from the battery of the portable terminal device which has a limited amount of electric energy.

According to still another aspect of the present invention, there is provided a program that causes the portable terminal device to perform the method for optical analysis.

The portable terminal device may include a tablet computer, a smartphone, a portable telephone, a personal computer, or other processing devices. The display unit may include a liquid crystal display device or an organic EL (electroluminescent) display device.

These and other objects, aspects and advantages of the present invention will become apparent to those skilled in the art from the following detailed description when read and understood in conjunction with the appended claims and drawings.

### BRIEF DESCRIPTION OF THE DRAWINGS

FIG. 1A schematically shows an exemplary configuration of an apparatus for optical analysis according to one embodiment of the present invention, with an external light source being attached to a processing device. The external light source is detachable from the processing device.

FIG. 1B schematically shows another exemplary configuration of an apparatus for optical analysis according to one embodiment of the present invention, with the external light source being attached to a microchip. The external light source is detachable from the microchip.

FIG. 2 schematically illustrates an exemplary configuration of an apparatus for optical analysis according to an embodiment of the present invention, with an external light source being built in the microchip.

FIG. 3A illustrates a second embodiment of the present invention. The second embodiment is a modification to the FIG. 2 embodiment. Similar to FIG. 2, the external light source is built in the microchip itself, but in FIG. 3 a chip for having the external light source therein and a chip for measuring light from the specimen are laminated.

FIG. 3B is a cross-sectional view taken along the line 3B-3B in FIG. 3A.

FIG. 4A schematically illustrates a step of taking out a specimen in an analyzing process according to an embodiment of the present invention.

FIG. 4B schematically illustrates a subsequent step of dropping and analyzing the specimen in the analyzing process according to the embodiment of the present invention.

FIG. 5 illustrates a third embodiment of the present invention. The external light source, which is similar to the one shown in FIG. 1A, is detachably attached to the processing device. Light from the external light source is introduced to the microchip via an optical fiber.

FIG. 6 shows an exemplary configuration of an external light source module that serves as the external light source.

FIG. 7A shows an optical fiber that is located at a position corresponding to a drive light introducing hole.

FIG. 7B shows an optical fiber that is located at another position which corresponds to a radiated light introducing hole.

FIG. 8 is a flowchart showing an exemplary process of analysis in a third embodiment of the present invention.

FIG. 9 is a cross-sectional view of a light introducing hole for a built-in camera.

FIG. 10 is a flowchart showing an exemplary process for optical analysis (optical measurement of a specimen solution positioned in a fluid passage, and operation/calculation to the measurement results).

FIG. 11 shows a fourth embodiment of the present invention. The external light source, which is similar to the one shown in FIG. 1B, is detachably secured to the microchip. Electricity is supplied to the external light source from the processing device such as a tablet computer.

FIG. 12 illustrates an exemplary configuration of an electricity feeding module configured to feed electric power to the external light source module which serves as the external light source mounted on (included in) the microchip.

FIG. 13 illustrates an exemplary configuration of the external light source module.

FIG. 14 illustrates a fifth embodiment of the present invention. The external light source, which is similar to the one shown in FIG. 2C, is built in the microchip. Electricity is supplied to the external light source from the processing device such as the tablet computer.

FIG. 15 is an electricity feeding module on a processing device side, which is one of the electricity feeding modules configured to feed electric power to the external light source built in the microchip.

FIG. 16 is an exemplary configuration of the external light source built in the microchip, and an electricity feeding module on a microchip side.

FIG. 17A depicts a chip in which a single external light source is build. The chip is used to measure light from the specimen.

FIG. 17B is a cross-sectional view taken along the line 17B-17B in FIG. 17A.

FIG. 17C is a cross-sectional view taken along the line 17C-17C in FIG. 17A.

FIG. 17D is a top view of the chip shown in FIG. 17A.

FIG. 18 illustrates an apparatus for optical analysis, which uses a configuration developed by the inventors (Japanese Patent Application No. 2013-35581) and described in the fourth embodiment (FIG. 11), together with a second light source or an external light source (electricity feeding module, external light source module) and a second introduced light guiding path for guiding light from the external light source.

FIG. 19 illustrates a modification to the analyzing apparatus of the third embodiment shown in FIG. 5, which does not include the built-in camera, the light introducing hole (inlet hole) for the built-in camera, and the light condensing hole, but includes a third lens and an observation hole.

FIG. 20 illustrates a modification to the analyzing apparatus of the fourth embodiment shown in FIG. 11, which does not include the built-in camera, and the light introducing hole for the built-in camera, but includes an observation hole.

FIG. 21A shows a modification to the microchip shown in FIG. 5, which includes a pre-treatment filter provided in the microchip.

FIG. 21B shows an enlarged view of the pre-treatment filter shown in FIG. 21A.

FIG. 22A shows a step of taking out a specimen in an analyzing process with the microchip.

FIG. 22B shows a subsequent step of dropping the specimen in the analyzing process with the microchip.

FIG. 22C shows an analyzing step, including various setting, controlling, data logging, and data sending and receiving, in the analyzing process with the microchip.

FIG. 23A shows a step of taking out a specimen in an analyzing process.

FIG. 23B shows a step of placing a microchip on a display unit of a processing device of an apparatus for optical analysis according to one embodiment of the present invention.

### DETAILED DESCRIPTION OF THE INVENTION

The inventors studied the problems of Patent Literature 1 and Non-Patent Literature 1, and developed a method and an apparatus for optical analysis, which can cope with various analyses, and use a disposal microchip to enable evaluation and analyses at a place where analysis needs to be conducted, in a short time and at high accuracy.

Firstly, relevant technologies developed by the inventors and difficulties encountered will be described. See, for example, Japanese Patent Application No. 2013-35581 or its Laid-Open Publication No. 2014-163818, published Sep. 8, 2014.

An optical analysis apparatus developed by the inventors includes a processing device and a microchip. The processing device includes a display unit (display portion) to display an image, and a control unit that possesses an operating/calculating function and a controlling function controlling the image to be displayed on the display unit. The microchip has a light inlet portion (introducing portion) and a light exit portion (emitting portion). The optical analysis is performed by placing the microchip on the display unit of the processing device, and introducing light into the microchip from the display unit.

The processing device is, for example, a tablet terminal device (tablet computer), a smartphone, a portable telephone, a personal computer or the like. In the following description, the processing device is a tablet computer. The display unit is, for example, a liquid crystal display unit, an organic EL display unit or the like.

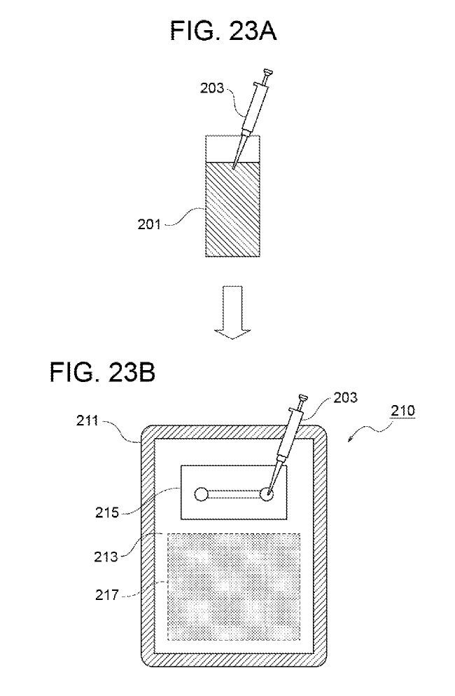

As shown in FIGS. 23A and 23B, therefore, the optical analysis apparatus 200 developed by the inventors includes a tablet computer 211 (processing device) having a display unit 213, and a microchip 215 placed on the display unit 213. A specimen (object to be analyzed) 201 is taken by a micropipette 203.

The specimen 201 is dropped from the micropipette 203 to a fluid passage of the microchip 215 on the display unit 213 of the tablet computer 211.

As a result, a reaction of the specimen 201 (e.g., a biomolecular reaction between an antigen and an antibody) takes place in the fluid passage of the microchip 215.

The microchip 215 is irradiated with light emitted from the display unit 213 of the tablet computer 211. With this irradiation of light, the reaction that takes place in the microchip 215 is measured. For example, when a light-induced fluorescence method is employed as a measuring method that uses the irradiation of light, fluorescence that corresponds to the reaction is observed.

Specifically, the light radiated from the display unit 213 is introduced to the light inlet portion of the microchip 215. The light is directed to the fluid, which contains the specimen 201 introduced to the fluid passage of the microchip 215 (referred to as “specimen-containing fluid”). Light (fluorescence) is observed from the specimen-containing fluid that is irradiated with the light. The observed light is taken out from the light exit portion of the microchip 215, and received by an image receiving unit (light receiving element) of the tablet computer 211 to detect the reaction of the specimen. Detection results are displayed on a display region 217 of the display unit 213.

It should be noted that the light emitted from the display unit 213 and introduced to the microchip 215 may be used to control the flow of the fluid, which contains the specimen 201 introduced to the microchip 215.

As described above, the apparatus for optical analysis developed by the inventors uses the light radiated from the display unit, which is originally designed to display an image, to detect the reaction of the specimen that takes place in the fluid passage of the microchip. For example, the reaction of the specimen (e.g., biomolecular reaction between the antigen and the antibody) inside the microchip (inside the fluid passage thereof), into which the specimen is introduced, is detected, as described above.

A liquid feeding unit (e.g., light-driven air pump) that is driven by light is provided to move (transport, convey) the liquid through the fluid passage of the microchip. Thus, the flow of the fluid, which contains the specimen introduced to the fluid passage of the microchip, is also controlled (regulated) by the light radiated from the display unit.

However, the intensity of the light radiated from the display unit of the tablet computer (processing device) is not always sufficient. If the intensity of the light radiated from the display unit of the tablet computer (processing device) is not sufficient, the intensity of the light introduced from the light inlet portion of the microchip and directed to the fluid (occasionally referred to as “radiated light” hereinafter), which contains the specimen introduced to the fluid passage of the microchip, is also weak. Then, the intensity of the light such as fluorescence (occasionally referred to as “observed light” hereinafter), which is observed from the specimen-containing fluid upon irradiation, also becomes weak. In some cases, the light from the specimen-containing fluid would be “not observable.”

The display unit of the processing device (e.g., the tablet computer, the smartphone, the portable telephone and the personal computer) cannot always emit light at a desired intensity in a desired wavelength range. For example, the relative spectral distributions of the light radiated from the display units of the tablet computers depend upon design specifications of tablet computer manufacturers, and therefore the relative spectral distribution of the light radiated from the display unit of the tablet computer made by one manufacturer is different from that of the tablet computer made by another manufacturer. Thus, one tablet computer can emit light from its display unit (display portion) at a sufficient intensity in a certain wavelength range, but may not be able to emit light from the display unit at a sufficient intensity in another wavelength range.

When the reaction that takes place in the fluid passage of the microchip is detected, a certain type of fluid may require light (e.g., ultraviolet light, infrared light, or perfect white light) at a wavelength different from the wavelength of the light that can be emitted from the display unit. In general, the light radiated from the display unit is continuous and incoherent light. However, the detection and measurement for the optical analysis may require other light than the light radiated from the display unit (light having different characteristics than the light radiated from the display unit). For example, the detection and measurement for the optical analysis may require pulsed light, coherent light, terahertz light or the like.

As such, a certain type of tablet computer is difficult to emit light from the display unit of the tablet computer at an intensity and wavelength that are suitable for the detection and measurement (observation) of the reaction of the specimen introduced to the fluid passage of the microchip. Also, it is difficult to cause the display unit of the tablet computer to emit pulsed light, coherent light and terahertz light, but such light (light having different characteristics from the light emitted from the display unit) may be required for detection and measurement (observation) of the reaction of the specimen.

The above-described facts are also true when light is used to drive a liquid feeding unit to move (convey) the liquid in the fluid passage. Specifically, a certain type of display unit is difficult to emit light with a sufficient intensity to drive the light-driven liquid feeding unit (e.g., light-driven air pump), or difficult to emit light with a sufficient intensity at a particular wavelength to drive the light-driven air pump when the light at that particular wavelength (wavelength suitable for driving the light-driven air pump) is required.

An object of the present invention is to provide a method for optical analysis, for use in an optical analyzing apparatus that can cope with various analyses, uses a disposal microchip, and can perform evaluation and analysis at high precision in a short time at a place where analysis is needed. The method uses a light source that can ensure light at a sufficient light intensity in a wavelength range suitable for at least optical analysis.

Now, embodiments of the present invention will be described in detail. It should be noted that the present invention is not limited to the illustrated and described embodiments. Various changes and modifications may be made to the embodiments without departing from the scope and spirit of the present invention. In the following description, redundant description may be omitted. Same or like reference numerals and symbols may be used in different drawings when such numerals and symbols designate the same or similar components. The optical analysis may include spectrophotometric analysis.

### First Embodiment

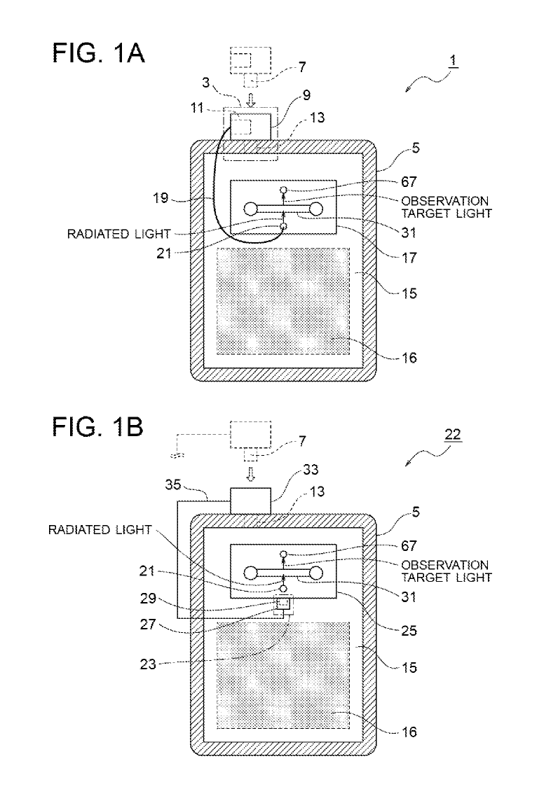

Referring to FIG. 1A, a light processing apparatus 1 according to a first embodiment of the present invention will be described. In FIG. 1A, an external light source 3 is connected to a processing device 5 such that the external light source 3 becomes integral with the processing device 5. The external light source 3 is detachable from the processing device 5. When the external light source 3 is attached to the processing device 5, the external light source 3 is electrically connectable to the processing device 5. A control unit of the processing device 5 controls the electric power to be fed to the external light source 3 from the processing device 5.

The external light source 3 is attached to or included in, for example, an external light source module 9 having a USB (Universal Serial Bus) terminal 7. The USB terminal 7 is electrically connected to the external light source 3. The external light source 3 includes, for example, an LED (Light Emitting Diode) 11.

The USB terminal 7 of the external light source module 9 is connected to a USB port 13 of the processing device 5. As a result, the external light source module 9 becomes integral with the processing device 5 via the USB port 13. The external light source module 9 is electrically connectable to the processing device 5 via the USB port 13 and the USB terminal 7.

A microchip 17 is placed on a display unit (display portion) 15 of the processing device 5 (e.g., tablet terminal device or a tablet computer). It should be noted that the microchip 17 may be placed on the display unit 15 or may be supported above the surface of the display unit 15 with a prescribed gap.

Light radiated from the external light source 3 is introduced to a light inlet portion 21 of the microchip 17 via a light guiding element such as an optical fiber 19. One end of the optical fiber 19 is fixedly secured to the external light source module 9, and the other end of the optical fiber 19 is coupled to the light inlet portion 21 of the microchip 17 via a coupling element (not shown). The other end of the optical fiber 19 is detachable from the light inlet portion 21 of the microchip 17. It should be noted that one end of the optical fiber 19 may be fixedly secured to the light inlet portion 21 of the microchip 17, and the other end of the optical fiber 19 may be attached to the external light source module 9 by a coupling element (not shown) such that the other end of the optical fiber 19 is detachable from the external light source module 9.

The external light source 3 (LED 11) is configured (selected) to emit light at an optimal wavelength with a sufficient optical intensity toward a fluid, which contains a specimen introduced in a fluid passage of the microchip 17.

FIG. 1B shows a schematic configuration of a light processing apparatus 22. An external light source 23 is detachably connected to a microchip 25. The external light source 23 is integral with the microchip 25. The external light source 23 is attached to or included in, for example, an external light source module 27. The external light source module 27 is detachable from the microchip 25. The external light source 23 includes, for example, an LED 29.

The LED 29 is configured (selected) to emit light at an optimal wavelength with a sufficient optical intensity toward a fluid, which contains a specimen introduced in a fluid passage 31 of the microchip 25.

Light radiated from the external light source is introduced to a light introducing portion 21 of the microchip 25.

Electricity is supplied to the external light source from an electric power feeding module 33, which is attached to the processing device 5, via an electric power feeding line 35. The power feeding module 33 is detachable from the processing device 5. When the power feeding module 33 is attached to the processing device 5, the power feeding module 33 is electrically connectable to the processing device 5. A control unit of the processing device 5 controls electricity to be fed to the power feeding module 33 from the processing device 5.

The power feeding module 33 has, for example, a USB terminal 7 and is connected to a USB port 13 of the processing device 5. As a result, the power feeding module 33 becomes integral with the processing device 5 via the USB port 13. The power feeding module 33 is electrically connectable to the processing device 5 via the USB port 13 and the USB terminal 7.

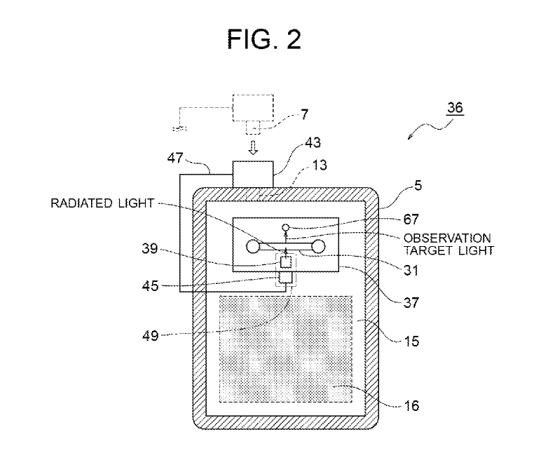

FIG. 2 schematically shows a light processing apparatus 36. An external light source is built in a microchip 37. The external light source which is built in the microchip 37 includes, for example, an LED 39. This is similar to the configurations shown in FIGS. 1A and 1B. The LED 39 is configured (selected) to emit light at an optimal wavelength with a sufficient optical intensity toward a fluid, which contains a specimen introduced in a fluid passage 31 of the microchip 37.

Electricity is supplied to the external light source from a power feeding module that includes a first power feeding module 43 on the processing device side and a second power feeding module 45 on the microchip side. The first power feeding module 43 is detachable from the processing device 5. The second power feeding module 45 is detachable from the microchip 37. When the power feeding module is attached to the processing device 5, the power feeding module is electrically connectable to the processing device 5. A control unit of the processing device 5 controls electricity to be fed to the power feeding module from the processing device 5.

The first power feeding module 43 on the processing device side has, for example, a USB terminal 7 and is connected to a USB port 13 of the processing device 5. As a result, the first power feeding module 43 becomes integral with the processing device 5 via the USB port 13. The first power feeding module 43 is electrically connectable to the processing device 5 via the USB port 13 and the USB terminal 7.

The second power feeding module 45 on the microchip side is electrically connected to the first power feeding module 43 via an electric power feeding line 47. When the second power feeding module 45 is connected to the microchip 37, the second power feeding module 45 is electrically coupled to the external light source (LED 39) which is built in the microchip 37.

Thus, the second power feeding module 45 on the microchip side and the external light source (LED 39) constitute the external light source module 49.

### Second Embodiment

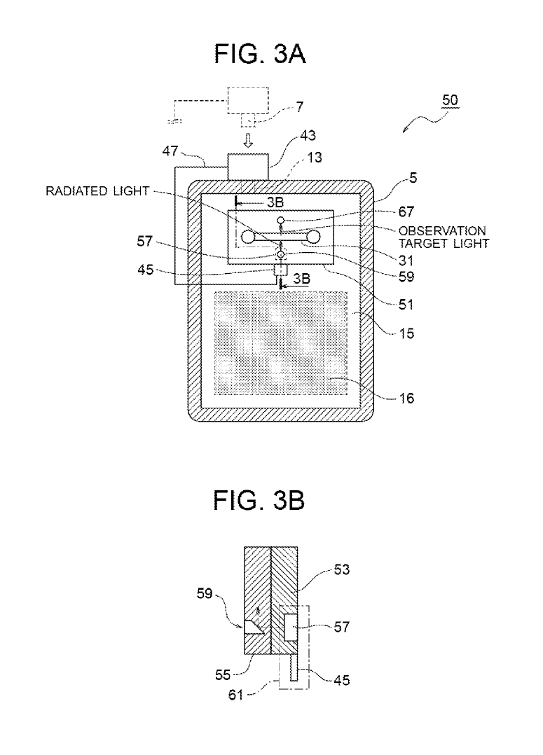

FIGS. 3A and 3B illustrate a modification to the configuration shown in FIG. 2. The external light source is built in the microchip in FIG. 2. FIG. 3A shows a schematic configuration of a light processing apparatus 50. In the configuration shown in FIG. 3A, a microchip 51 has a two-layer structure, which includes a first chip 53 and a second chip 55 laminated on the first chip 53. The first chip 53 has an external light source therein. The second chip 55 is used to measure light emitted from a specimen.

The external light source 57 is included in (built in) the first chip 53. The external light source 57 includes, for example, an LED. This configuration is similar to FIG. 2. The LED is configured (selected) to emit light at an optimal wavelength with a sufficient optical intensity toward a fluid, which contains a specimen, introduced in a fluid passage of the microchip 51.

As shown in FIG. 3B, the microchip 51 has the first chip 53 for the external light source, and the second chip 55 for measurement of specimen radiation. The second chip 55 is situated on the first chip 53. Light from the external light source 57 (LED) of the first chip 53A is introduced to an introducing portion 59 of the second chip 55.

Electricity is supplied to the external light source 57 from a power feeding module, which includes a first power feeding module 43 on the processing device side, and a second power feeding module 45 on the microchip side. This is similar to the configuration shown in FIG. 2. When the power feeding module is attached to the processing device 5, the power feeding module is electrically connectable to the processing device 5. A control unit of the processing device 5 controls the electric power to be fed to the power feeding module from the processing device 5.

The first power feeding module 43 on the processing device side has, for example, a USB terminal 7, and is connected to a USB port 13 of the processing device 5. As a result, the first power feeding module 43 becomes integral with the processing device 5 via the USB port 13. The first power feeding module 43 is brought into a condition that the first power feeding module 43 is electrically connectable to the processing device 5 via the USB port 13 and the USB terminal 7.

The second power feeding module 45 on the microchip side is electrically connected to the first power feeding module 43 via a power feed line 47. When the second power feeding module 45 is attached to the external light source chip (chip in which the external light source is built) 53 of the microchip 51, the second power feeding module 45 is electrically connected to the external light source 57 (LED) in the external light source chip 53.

Thus, the second power feeding module 45 and the external light source 57 (LED) built in the external light source chip 53 constitute in combination the external light source module 61.

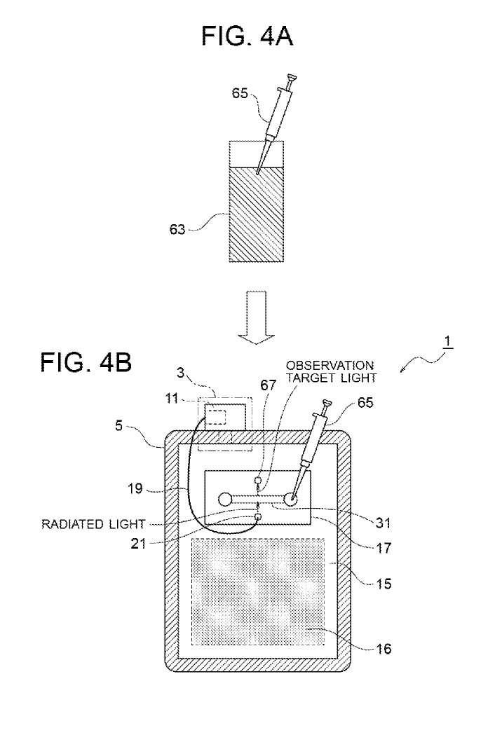

FIGS. 4A and 4B schematically show the analyzing process according to the embodiment of the present invention. The light processing apparatus 1 of the first embodiment, as shown in FIG. 1A, is used in the following description.

Referring to FIG. 4A, firstly, a necessary amount of specimen 63 (object to be analyzed) is taken out by a micropipette 65. The specimen 63 is derived from, for example, a human body, an animal, river or wasted liquid. If necessary, a pretreatment, such as removing impurities or filtering, may be performed before the specimen 63 is taken out by the micropipette 65.

Subsequently, the specimen 63 is dropped from the micropipette 65 into a fluid passage 31 of a microchip 17 placed on a display unit (display portion) of a tablet computer (processing device 5).

As a result, a reaction of the specimen 63 takes place in the microchip (in the fluid passage 31 thereof). The reaction is, for example, a biomolecular reaction between an antigen and an antibody.

The microchip 17 is irradiated with light from the external light source 3 via a fiber 19. The light is guided by the fiber 19. The external light source 3 includes, for example, an LED 11, and emits light at an optical intensity and wavelength that meet conditions suitable for desired measurement (will be described).

With this irradiation, the reaction that takes place in the microchip is measured. For example, when the light-induced fluorescence measuring method that uses the radiated light is employed to measure the reaction, fluorescence that corresponds to the reaction is observed.

The microchip 17 has a light introducing (inlet) portion 21 and a light emitting (exit) portion 67, and receives light that comes from the external light source 3 via the fiber 19, at the light introducing portion 21. Light received by the microchip 17 is directed to the fluid, which contains the specimen 63 introduced to the microchip 17, and causes the specimen-containing fluid to emit light. The light radiated from the specimen-containing fluid is guided to the outside from the light emitting portion 67.

It should be noted that a plurality of external light sources may be provided. For example, a second external light source may be provided in addition to the above-described external light source 3, and light emitted from the second external light source is guided by an optical fiber and used to control (regulate) the flow of the fluid, which contains the specimen 63 introduced to the microchip 17.

When the light emitted from the specimen upon reaction in the microchip 17 is visible light, it is possible to confirm light emission (or no light emission) by visual inspection (observation by human eyes), and in turn to confirm presence (or absence) of the reaction in the microchip.

On the other hand, when a light receiving element (e.g., a camera) is built in the tablet computer 5, the light emitted from the specimen upon the reaction in the microchip 17 is guided to the light receiving element. Then, the reaction is detected at (by) the light receiving element of the tablet computer 5.

Automatic positioning or the like may be carried out if a resolving power of the camera is utilized. A dispersing element may be provided in the microchip 17 in order to measure a spectrum of a signal light. A detection signal generated from the light receiving element is operated and calculated by an operating and calculating unit of the tablet computer. The operating and calculating unit processes the detection signal for analysis, and also displays the analysis result in the display area 16 of the display unit 15 (FIG. 4B). The operating and calculating unit also perform the logging in of the data, and the transmission of the data.

A set of software used to analyze (process, operate and calculate) the detection signal may be downloaded from outside depending upon analysis contents to be applied to the specimen. The downloading may be performed by using a communication function of the tablet computer 5. Appropriate software may be selected from the set of downloaded software when the measurement and analysis are conducted, depending upon analysis items to be applied to the measurement target. The downloaded set of programs (software) for analysis may be updated to a new version of programs at an appropriate timing by using the communication function of the tablet computer 5.

### Usefulness of the Analyzing Apparatus

The analyzing apparatus uses, as an analyzer (processing device), a portable display device (tablet computer) that has an operating and calculating function. The analyzing apparatus uses a microreactor that includes the microchip placed on the display device to, for example, isolate, synthesize, extract, and analyze a trace amount of reagent or specimen.

Because the tablet computer that has an electric power feeding port (power supply port) is used, light emitted from the external light source that receives electricity from the electric power feeding port can be used as light to detect the analysis target (object be analyzed) and/or as an energy to drive relevant components. The operating and calculating unit, which is built in the tablet computer, can be used to operate, calculate and analyze the detection signal obtained from the analysis target. It is also possible to display the analysis results on the display device.

The external light source may include an LED (or LEDs), or an LD (or LDs). LD stands for laser diode. By appropriately selecting (deciding) the intensity of the light emitted from the external light source and/or spectral characteristics of the emitted light, it is possible to use light that has a wavelength and intensity suitable for observation of the reaction of the specimen introduced to the fluid passage of the microchip. By appropriately selecting (deciding) the characteristics of the light (e.g., pulsed light or coherent light) emitted from the external light source, it is possible to use light that has a characteristic suitable for observation of the reaction of the specimen introduced to the fluid passage of the microchip. The external light source and the power feeding module, which has a power feeding terminal electrically coupled to the external light source, constitute in combination the external light source module. By electrically coupling the power feeding module to the power feeding port of the tablet computer, it is possible to feed electricity to the external light source module from the tablet computer.

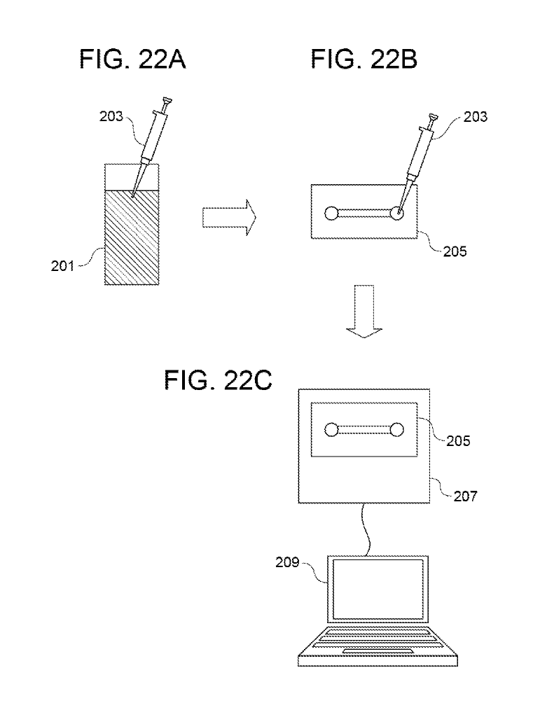

Thus, the analyzing apparatus of this embodiment integrates the external light source module and the operating/calculating device in (on) the portable tablet terminal device (tablet computer). The analyzing apparatus uses the tablet terminal device and the microchip, which does not include an active element (component), to carry out the analysis. Unlike the conventional apparatus, the analyzing apparatus of this embodiment does not need a dedicated control device (see FIG. 22C). As such, the analyzing apparatus of this embodiment is compact and portable, and can perform the evaluation and analysis at high precision in a short testing time at a site where the analysis is needed. Accordingly, the analyzing apparatus of this embodiment can meet the demand for the POCT (point of care testing) in, for example, the life science technology.

The tablet terminal device serves as the detection system, and therefore the microchip can be an inexpensive ordinary chip that does not include an active element. Thus, the microchip is disposal. For example, the microchip does not have to be integral with a detection system that has an integrated organic EL element, and therefore the microchip does not have to be expensive.

When the external light source 3 is attached to the tablet terminal device as shown in FIG. 1A, the light from the external light source 3 is guided to the microchip 17 by the optical fiber 19. When the external light source 23 is attached to the microchip 25 as shown in FIG. 1B, the light from the external light source 23 is directly introduced to the microchip 25. When the external light source is built in the microchip 37, 51 as shown in FIGS. 2 and 3A, the light from the external light source is directly introduced to the light introducing portion of the microchip 37, 51. Thus, even if the microchip 37, 51 is displaced to a certain extent on the tablet terminal device, such displacement does not affect the optical analysis. Because the external light source is built in the microchip 37, 51, alignment between the external light source and the light introducing portion of the microchip 37, 51 is not necessary. Accordingly, the position adjustment of the microchip 37, 51 on the tablet terminal device is unnecessary, and the measurement can be carried out quickly.

As described above, the analyzing apparatus of this embodiment uses the communication function of the tablet terminal device to appropriately download the software for analysis, depending upon the analysis contents to be applied to the measurement target. Thus, the analyzing apparatus can perform various analyses to various specimens.

Because the single analyzing apparatus can cope with various analyses, it is not necessary to prepare many analyzing apparatuses to cope with various analyses. The conventional analyzing apparatus is customized for a particular analysis, and therefore many analyzing apparatuses are necessary to cope with various analyses.

The analyzing apparatus of the above-described embodiment can easily log the measurement data in the tablet terminal device (data logging to the tablet terminal device). Thus, the analyzing apparatus does not need a dedicated storage for the measurement data. Also, it is easy to establish an analyzing system using the communication function. Items displayed on the display unit may be customized depending upon functions such as selection of emission colors, and indication of analysis data.

It should be noted that the processing device used for the present invention is not limited to the tablet terminal device. For example, a personal computer, a smartphone, a mobile telephone, or other electronic device may be used instead of the tablet terminal device as long as it is a processing device having a display unit (display portion) and possesses an operating and calculating function.

### Third Embodiment

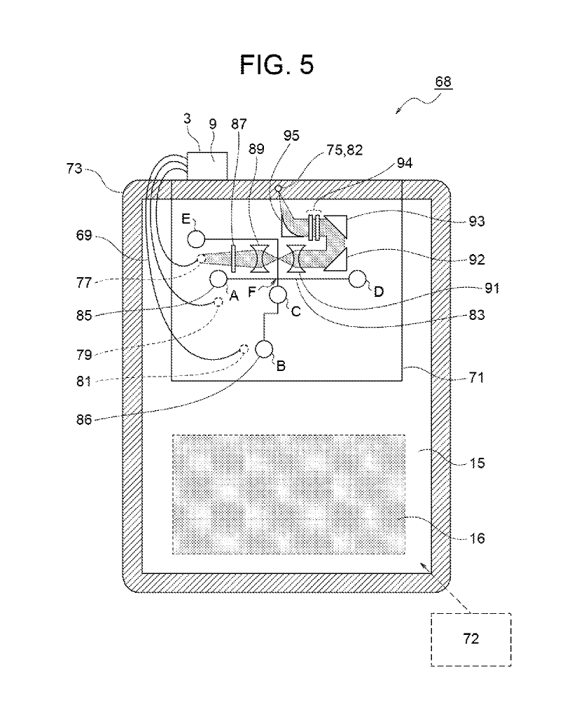

Referring to FIG. 5, a third embodiment of the present invention will be described. FIG. 5 schematically illustrates a configuration of a light processing apparatus 68. Similar to the embodiment shown in FIG. 1A, the external light source is attached to the processing device in this embodiment. The external light source is detachable from the processing device. Light from the external light source is introduced to the microchip via an optical fiber.

In the third embodiment, the processing device that has the display unit 15 and the control unit 72 is a portable tablet terminal device 73. The tablet terminal device 73 has a built-in camera 75 as the light receiving element. The microchip 71 is placed on the surface of the tablet terminal device such that it extends over an area including part of the display unit and the built-in camera 75. It should be noted that the microchip 71 may be supported above the above-mentioned area of the surface of the tablet terminal device with a small gap. For example, the microchip 71 may be situated 1 mm above the surface of the tablet terminal device.

It should also be noted that for the sake of easier understanding the size of the microchip 71 is exaggerated in FIG. 5. Thus, the real size relationship between the tablet terminal device 73 and the microchip 71 may be different from what is depicted in FIG. 5.

The microchip 71 is made from, for example, silicone resin such as PDMS (Polydimethylsiloxane). As shown in FIG. 5, the microchip 71 has, at least, a light introducing portion (e.g., hole 77 for introducing radiated light, hole 79 for introducing driving light, and hole 81 for introducing driving light), a light emitting portion (e.g., hole 82 for introducing light to the built-in camera 75), a plurality of ports A-E for retaining the specimen-containing liquid, and/or buffer solution, a fluid passage (micro fluid passage 83) connecting the ports A-E, liquid conveying units (e.g., light-driven air pump 85) provided at the ports for sending (transporting) the liquid, which is retained in the associated port, a light guiding path (path defined by the filter 87, the first lens 89 and other components) for guiding the light from the light introducing portion and irradiating the specimen-containing liquid with the light, and another light guiding path (path defined by the second lens 91, the first parallel light filter 92, the second parallel light filter 93 and other components) for guiding light, which is emitted from the specimen upon irradiating the specimen-containing liquid with the light, to the light emitting portion. The liquid conveying units are driven by light.

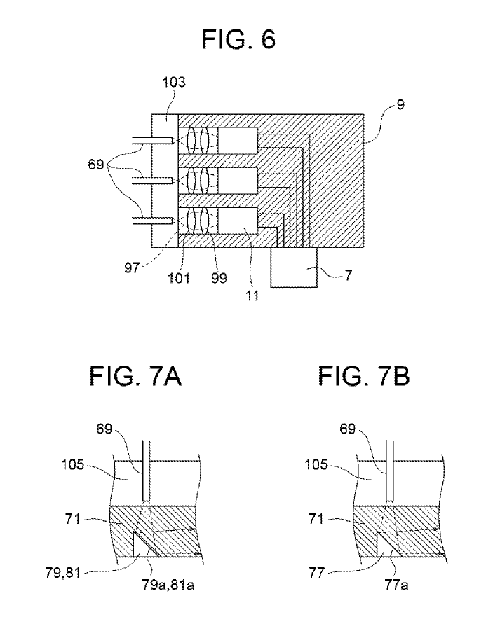

FIG. 6 shows an exemplary configuration of the external light source module 9 that includes the external light source 3. The external light source module 9 shown in FIG. 6 has, for example, an electric power feeding terminal (i.e., USB terminal 7). As the USB terminal 7 is engaged into the USB port (power feeding port) of the tablet terminal device 73 (i.e., processing device), it becomes possible to feed electricity to the external light source module 9 from the tablet terminal device 73.

The external light source module 9 of FIG. 6 has three light sources therein. Each of the three light sources has an LED 11. Light emitted from the three LEDs 11 is introduced to the three holes 77, 79 and 81 of the microchip 71 via the associated optical fibers 69 respectively (will be described later).

The LED 11 which corresponds to the radiated light introducing hole 77 emits light that has an optimal wavelength with a sufficient light intensity, as the light to be directed to the fluid which contains the specimen introduced to the fluid passage of the microchip 71. The LEDs 11 which correspond to the driving light introducing holes 79 and 81 emit light that has an optimal wavelength with a sufficient light intensity as the light to drive the light-driven air pump 85.

On the light emitting side of each LED 11, there is provided a light condensing hole 97. A collimator lens 99 and a condensing lens 101 are disposed in the condensing hole 97 in this order from the LED 11 side.

Light emitted from each LED 11 is incident to the associated collimator lens 99 for collimation. Then, the collimated light is incident to the condensing lens 101. The light incident to the condensing lens 101 is condensed to an end face of the associated optical fiber 69. The three optical fibers 69 are positioned (supported in position) by an optical fiber manifold 103 such that the light from the three LEDs 11 is condensed and introduced to the three optical fibers 69 respectively.

Referring back to FIG. 5, the light-driven air pumps (micropumps) 85 and 86 are provided in the ports A and B of the microchip, respectively. The light-driven air pumps 85 and 86 may be those which are disclosed in Patent Literature 2. For example, the light-driven air pump 85, 86 has a gas generating chamber to receive a gas generating agent which generates a gas when it is irradiated with light. The gas generated upon irradiation causes the fluid to move in the fluid passage.

The light-driven air pumps are driven upon introducing the light to the ports A and B from the LEDs 11 of the external light source module 9, respectively.

The functions and roles of the respective ports A to E shown in FIG. 5 are described below.

The port A is a reservoir of liquid (liquid receiver). The light-driven air pump 85 is provided at the port A. The specimen is introduced to the port A. The light-driven air pump 85 may be referred to as a “first light-driven air pump.”

The port B is a port at which another light-driven air pump 86 is provided. The light-driven air pump 86 may be referred to as a “second light-driven air pump.”

The port C is a reservoir of liquid (liquid receiver) to receive and reserve, for example, a buffer solution (PBS or phosphate buffered saline)

The port D is a reservoir of the specimen.

The port E is an exit (outlet) of the specimen.

In this embodiment, the analysis on the specimen introduced to the microchip 71 will be carried out in the following manner.

The three optical fibers 69, which guide the light from the external light source module 9 of FIG. 6, are connected to three positions that respectively correspond to the three light introducing portions (holes 77, 79 and 81) of the microchip 71. FIG. 7A shows the optical fiber 69 connected to the hole 79, 81 for introducing the driving light. FIG. 7B shows the optical fiber 69 connected to the hole 77 for introducing the radiated light.

As depicted in FIGS. 7A and 7B, the light introducing holes 79, 81 and 77 are formed in that surface of the microchip 71 which is opposite the associated optical fibers 69. This surface of the microchip 71 is referred to as the lower face or back face of the microchip 71. Each of the light introducing holes 79, 81 and 77 has an inclined wall 79a, 81a, 77a, which has a predetermined angle relative to the lower surface of the microchip 71. This inclined wall (inclined surface) 79a, 81a, 77a extends to the bottom of the light introducing hole from the lower surface of the microchip 71. In other words, it can be said that the bottom wall 79a, 81a, 77a of the light introducing hole is inclined, and extends to the lower surface of the microchip 71.

Each of the optical fibers 69 extending from the external light source module 9 is supported and positioned by an optical fiber holder 105 such that the light emitted from the optical fiber 69 proceeds through the microchip 71 and reaches the inclined surface 79a, 81a, 77a of the associated light introducing hole 79, 81, 77.

As described above, the microchip 71 is made from silicone resin such as PDMS. In general, the refractive index of the silicone resin is greater than the refractive index of the air (atmosphere). Thus, when the light emitted from each optical fiber 69 is incident to the inclined wall 79a, 81a, 77a of the light introducing hole 79, 81, 77 at an incident angle that is equal to or greater than a critical angle of the microchip 71 (silicone resin) to the air, then the light is totally reflected by the inclined wall 79a, 81a, 77a. By appropriately deciding the angle of the inclined wall 79a, 81a, 77a, the light emitted in the vertical direction to the surface of the microchip 71 from each optical fiber 69 is reflected (turned) to a transverse direction (lateral direction) by the inclined wall 79a, 81a, 77a of the light introducing hole.

The inclined wall 79a, 81a of the driving light introducing hole 79, 81 is formed such that the light turned by the inclined wall 79a, 81a is directed to the associated light-driven air pump 85, 86 disposed at the port A, B. Likewise, the inclined wall 77a of the radiated light introducing hole 77 is formed such that the light turned by the inclined wall 77a is directed toward a first lens 89 (will be described). As such, the light introduced to the microchip 71 from the external light source module 9 via the optical fibers 69 drives the liquid feeding (conveying) units (e.g., light-driven air pumps 85 and 86) to control the flow of the liquid, which contains the specimen introduced to the microchip 71. Also, the specimen-containing fluid is irradiated with this light to cause the specimen-containing liquid to emit light such that the emitted light is used for the analysis of the specimen.

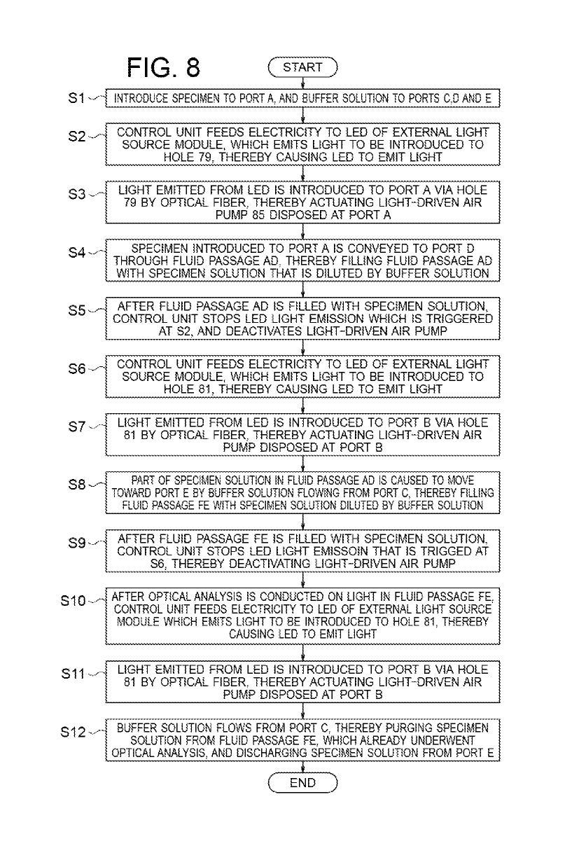

Referring now to FIG. 8, the analyzing process of this embodiment will be described. In this embodiment, the specimen introduced to the microchip 71 moves in the fluid passage in the microchip 71 and undergoes the analyzing process in the manner shown in FIG. 8.

Firstly, the specimen is introduced from the port A. The buffer solution (e.g., PBS) is introduced from the ports C, D and E at Step S1.

Then, the control unit 72 which is built in the tablet terminal device 73 feeds the electric power to that LED in the external light source module 9 which emits light to be introduced to the light introducing hole 79, to cause the LED to emit light (Step S2).

The light emitted from the LED is introduced to the light introducing hole 79 by the associated optical fiber 69, and turned by the inclined wall 79a of the light introducing hole 79 such that the light proceeds to the port A. Thus, the light-driven air pump 85 disposed at the port A is driven (Step S3).

As the light-driven air pump 85 is driven, the specimen introduced to the port A at Step S1 is caused to move in the fluid passage AD (fluid passage between the ports A and D) toward the port D. In the fluid passage AD, the specimen introduced to the port A meets the buffer solution (PBS) introduced to the port D such that the fluid passage AD is filled with the solution of the specimen that is diluted by the buffer solution (PBS) (Step S4).

It should be noted that the buffer solution may be introduced to the port A and the specimen may be introduced to the port D in Step S1. In Step S4, the light-driven air pump 85 may be driven such that the buffer solution may be conveyed from the port A toward the specimen at the port D.

After the fluid passage AD is filled with the solution of the specimen that is diluted by the buffer solution (PBS) at Step S4, the control unit 72 of the tablet terminal device 73 stops feeding the electric power to the LED that emits the light to be introduced to the light introducing hole 79. Accordingly, the control unit 72 stops the light emission of the LED, and deactivates the light-driven air pump 85 (Step S5).

Subsequently, the control unit 72 of the tablet terminal device 73 feeds the electric power to that LED in the external light source module 9 which emits light to be introduced to the light introducing hole 81, to cause the LED to emit light (Step S6). The light emitted from the LED is introduced to the light introducing hole 81 by the associated optical fiber 69, and turned by the inclined wall 81a of the light introducing hole 81 such that the light proceeds to the port B. Thus, the light-driven air pump 86 disposed at the port B is driven (Step S7).

As the light-driven air pump 86 is driven, the gas generated at the port B and the buffer solution introduced to the port C at Step S1 are caused to move in the fluid passage CE (fluid passage 10 between the ports C and E) toward the port E. Therefore, the solution of the specimen, which is diluted by the buffer solution (PBS) and present at the intersection F of the fluid passages CE and AD, is caused to move toward the port E from the intersection F by the buffer solution flowing from the port C. Accordingly, part of the solution of the specimen diluted by the buffer solution (PBS) in the fluid passage AD is caused to move toward the port E together with the buffer solution flowing from the port C. In other words, the buffer solution flowing from the port C forces that part of the solution of the specimen to move toward the port E. This part of the solution of the specimen meets the buffer solution (PBS) introduced to the port E in the fluid passage FE (fluid passage between the intersection F and the port E) such that the fluid passage FE is filled with the solution of the specimen diluted by the buffer solution (PBS) (Step S8).

After the fluid passage FE is filled with the solution of the specimen diluted by the buffer solution (PBS) at Step S8, the control unit 72 of the tablet terminal device 73 stops feeding the electric power to the LED that emits the light to be introduced to the light introducing hole 81. Accordingly, the control unit 72 stops the light emission of the LED, and deactivates the light-driven air pump 86 (Step S9).

The optical analyzing unit (will be described) conducts the optical analysis on the light in the fluid passage FE. Then, the control unit 72 of the tablet terminal device 73 feeds the electric power to that LED in the external light source module 9 which emits light to be introduced to the light introducing hole 81, to cause the LED to emit light (Step S10). The light emitted from the LED is introduced to the light introducing hole 81 by the associated optical fiber 69, and turned by the inclined wall 81a of the light introducing hole 81 such that the light proceeds to the port B. Thus, the light-driven air pump 86 disposed at the port B is driven (Step S11). As the light-driven air pump 86 is driven, the buffer solution is caused to move from the port C, as described above. Thus, the solution of the specimen in the fluid passage FE, which already underwent the optical analysis, is purged by the buffer solution (more precisely, a flesh solution of the specimen, which is diluted by the buffer solution (PBS) and does not yet undergo the optical analysis, and which contains the solution of the specimen derived from the intersection F) such that the solution of the specimen is discharged from the port E (Step S12).

As shown in FIG. 5, the optical analyzing unit used to carry out the optical analysis on the light in the fluid passage FE includes, for example, the radiated light introducing hole 77, a filter 87, the first lens 89, a second lens 91, two parallel light filters (first filter 92 and second filter 93), a filter 94, a light condensing hole 95 and a light introducing hole 82 for the built-in camera 75.

As described above, the included surface 77a of the light introducing hole 77 is configured to guide the light toward the first lens 89. The light emitted from the LED 11 and guided through the optical fiber 69 is diffused light, and therefore a certain component of the light proceeding in the transverse direction (light proceeding toward the first lens 89) spreads in the vertical direction, and is incident to the upper surface of the microchip 71. The light incident to the upper surface of the microchip 71 is reflected at the interface between the microchip 71 and the air and guided toward the first lens 89 if the incident angle is equal to or greater than the critical angle of the microchip 71 (silicone resin) to the air.

Thus, the optical path in the microchip 71 (silicone resin) through which the light proceeds from the light introducing hole 77 to the first lens 89) serves as the waveguide (light guiding path) for the light emitted from the LED 11.

It should be noted that the filter 87 is disposed in the optical path between the light introducing hole 77 and the first lens 89. The light introduced to the light introducing hole 77 from the LED 11 is used as the light to excite the specimen in the fluid passage FE.

The LED 11 that emits the light to be introduced to the light introducing hole 77 is selected such that the light has a wavelength suitable for excitation of the specimen. The external light source module 9 includes such LED 11. The light emitted from the LED 11 has a relatively wide spectral line width. Accordingly, the light emitted from the LED 11 may contain a wavelength component that is not necessary for the excitation of the specimen. The unnecessary wavelength component in the light may cause errors in the measurement. The filter 87 removes (cuts off) such unnecessary wavelength component from the light emitted from the LED 11.

The first lens 89 is defined by a hollow space formed in the microchip 71. The incident surface of the light emitted from the LED of the first lens 89 is concave, and the exiting surface of the light is also concave. The concave shape of the light incident surface and the concave shape of the light exiting surface are decided such that the light passing through the first lens 89 is incident to the fluid passage FE of the microchip 71 and condensed in the fluid passage FE.

Thus, the hole 77 for introducing the radiated light reflects the light emitted from the LED 11 and guided by the optical fiber 69 toward the filter 87 and the first lens 89. The reflected light proceeds in the microchip 71, which is made from the silicone resin for example, and is incident to the filter 87 and the first lens 89. The light emitted from the LED 11 and incident to the first lens 89 via the filter 87 is condensed in the fluid passage FE by the first lens 89, and the solution of the specimen diluted by the buffer solution (PBS) in the fluid passage FE is excited by the light.

As the specimen is excited by the light, the specimen emits light (e.g., fluorescence) depending upon the physical property of the specimen. This light proceeds through the second lens 91, two parallel light filters (first filter 92 and second filter 93), a filter 94, a condensing hole 95 and the light introducing hole 82 for the built-in camera 75 in this order, and is incident to the built-in camera 75 of the tablet terminal device 73 such that the light is detected by the built-in camera 75. This light is light to be observed, and referred to as “observation target light.”

The second lens 91 is defined by a hollow space formed in the microchip 71. The incident surface of the observation target light of the second lens 91 and the exiting surface of the observation target light have curved shapes such that the exiting light from the second lens 91 becomes parallel light.

The first parallel light filter 92 is defined by a hollow space formed in the microchip 71. The hollow space has a shape of, for example, a triangular prism. The parallel light that exits the second lens 93 is incident to the inclined surface of the triangular filter 92. The inclined surface of the triangular filter 92 is inclined 45 degrees relative to the optical axis of the parallel light. When the material of the microchip 71 is PDMS that has a refractive index of 1.41, then the critical angle of the PDMS is approximately 45 degrees. Thus, the incident angle of the parallel light to the inclined surface of the triangular filter 92 is almost equal to the critical angle, and the parallel light incident to the inclined surface of the filter 92 is totally reflected in the 90-degree direction (at right angles) (upward in FIG. 5).

The second parallel light filter 93 is defined by a hollow space formed in the microchip 71. The hollow space has a shape of, for example, a triangular prism. The parallel light from the first parallel filter 92 is incident to the inclined surface of the triangular filter 93. The inclined surface of the triangular filter 93 is inclined 45 degrees relative to the optical axis of the parallel light. Similar to the first parallel light filter 92, the incident angle of the parallel light to the inclined surface of the second parallel light filter 93 is almost equal to the critical angle, and the parallel light incident to the inclined surface of the filter 93 is totally reflected in the 90-degree direction (at right angles) (leftward in FIG. 5).

As described above, when the material of the microchip 71 is the PDMS, the critical angle of each of the first parallel light filter 92 and second parallel light filter 93 is approximately 45 degrees. Thus, each of the first and second parallel light filters 92 and 93 totally reflects that component of the incident light which has an incident angle equal to or greater than 45 degrees.

The light incident plane of each of the first and second parallel light filters 92 and 93 is inclined 45 degrees relative to the optical axis of the incident parallel light. Thus, when the light is incident to the first parallel light filter 92 at the incident angle greater than 45 degrees, the light is totally reflected by the first parallel light filter 92, but the incident angle of the light becomes smaller than 45 degrees when the light is incident to the second parallel light filter 93. Accordingly, this light is not reflected by the second parallel light filter 93, but passes through the hollow space of the second parallel light filter 93.

Because the parallel light filters (first filter 92 and the second filter 93) are configured and arranged in the above-described manner, the parallel light filters only filter out that component of the light incident to the parallel light filters which is not the parallel component, and only allow the parallel component of the light to reach the filter 94.

The light exiting the parallel light filter 93 and incident to the filter 94 may include not only the observation target light (e.g., fluorescence) emitted from the specimen but also other light. Specifically, the radiated light that does not contribute to the excitation of the specimen may also pass through the parallel light filters 92 and 93 and be incident to the filter 94. This radiated light becomes a noise to the optical analysis of the specimen, and therefore it should be removed.

The filter 94 cuts off (filters out) the radiated light component from the light exiting the parallel light filter 93. The filter 94 may be a dielectric optical element (notch filter) embedded in the microchip 71. This is a cutting off configuration. Alternatively, the filter 94 may be a dye (pigment, colorant) embedded in the microchip 71 to absorb the radiated light component. This is an absorbing configuration.

The condensing hole 95 is defined by a cavity formed in the microchip 71. The cavity has, for example, a cylindrical shape. The light incident face of the condensing hole (cavity) 95 for the light exiting the filter 94 reflects and condenses the incident light. Also, the light incident face of the light condensing hole 95 is configured such that the optical axis direction of the reflected and condensed light extends substantially perpendicularly to the optical axis of the incident light. In the example shown in FIG. 5, the light condensing hole 95 is configured to condense the reflected light to (at) the light introducing hole 82 for the built-in camera 75.

In order for the condensing hole 95 to efficiently reflect the incident light, the shape of the reflecting surface of the condensing hole 95 is decided in consideration of the critical angle of the interface (reflecting surface) that depends upon the material of the microchip 71.

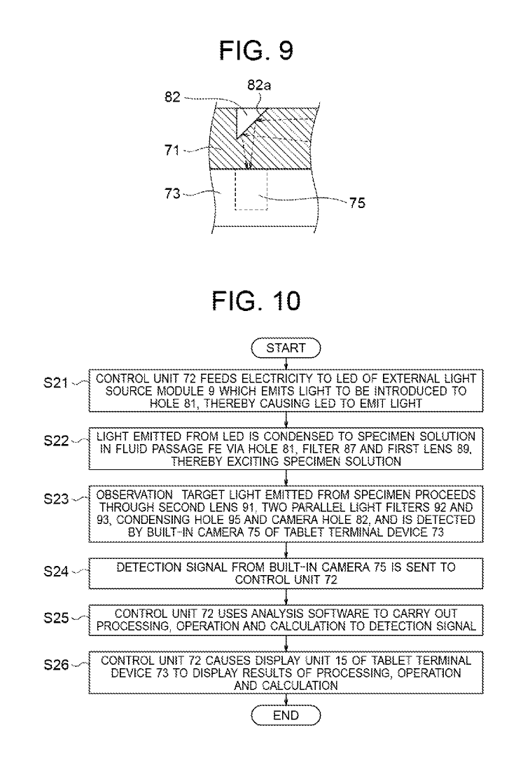

FIG. 9 shows the cross-section of the light introducing hole 82 for the built-in camera 75. The light introducing hole 82 for the built-in camera 75 has an inclined wall 82a. This inclined wall (inclined surface) 82a is inclined relative to the surface of the microchip 71. The inclined wall 82a extends to the surface of the microchip 71 from the bottom of the light introducing hole 82. In other words, it can be said that the bottom wall 82a of the light introducing hole 82 is inclined, and extends to the surface of the microchip 71.

The observation target light, which is condensed by the condensing hole 95, is incident to the bottom wall of the light introducing hole 82 for the built-in camera 75. As described above, the angle of the inclined wall 82a is appropriately decided in consideration of the critical angle and other sizes and shapes of the microchip 71, and therefore the observation target light condensed by the condensing hole 95 is turned downward (in the direction toward the built-in camera 75) by the inclined wall 82a, and condensed to the built-in camera 75.

In this manner, the observation target light (e.g., fluorescence) from the specimen and the radiated light that does not contribute to the excitation of the specimen are collimated by the second lens 91, the parallel light is only extracted by the two parallel light filters 92 and 93, the parallel light is incident to the filter 94, the radiated light component is cut off by the filter 94, the remaining light is condensed by the condensing hole 95 and introduced to the light introducing hole 82 for the built-in camera 75, and this light is introduced to the built-in camera 75 of the tablet terminal device 73 by the light introducing hole 82. Accordingly, the observation target light is detected by the built-in camera 75.

### Optical Measurement and Operation/Calculation in the Tablet Terminal Device: Optical Analysis

Between Steps S9 and S10 in FIG. 8, the optical analysis is carried out on the light in the fluid passage FE. The optical analysis includes the optical measurement for the solution of the specimen in the fluid passage FE, and the operation and calculation to the measurement results.

The optical analysis is carried out in the manner, for example, as shown in FIG. 10.

Firstly, the control unit 72 of the tablet terminal device 73 feeds the electricity to that LED of the external light source module 9 which emits light to be introduced to the light introducing hole 81 from the external light source module 9, so as to cause this LED to emit light (Step S21 in FIG. 10). The light emitted from this LED (radiated light) includes a wavelength component that is suitable for the optical measurement for the solution of the specimen present in the fluid passage FE. In other words, the radiated light includes a light component suitable for excitation of the specimen.

The light emitted from the LED and introduced to the light introducing hole 81 is condensed in the fluid passage FE via the filter 87 and the first lens 89. Thus, the radiated light having a wavelength suitable for the excitation of the specimen is condensed to the solution of the specimen in the fluid passage FE to excite the solution of the specimen (Step S22).

The observation target light emitted from the excited specimen passes through the second lens 91, the two parallel light filters 92 and 93, the filter 94, the condensing hole 95 and the camera light introducing hole 82 in this order, and is incident to the built-in camera 75 of the tablet terminal device 73 such that the observation target light is detected by the built-in camera 75 (Step S23).

A detection signal that represents the observation target light detected by the built-in camera 75 is sent to the control unit 72 of the tablet terminal device 73 from the built-in camera 75 (Step S24).

Upon receiving the detection signal from the built-in camera 75, the control unit 72 uses the software for analysis, which is stored in the tablet terminal device 73 beforehand or downloaded from outside via the communication function of the tablet terminal device 73, to carry out the operation and calculation to the detection signal (Step S25).

The control unit 72 causes the display unit 15 to display the results of the processing and the results of the operation and calculation in the analysis result display area 16 of the display unit 15 of the tablet terminal device 73 (Step S26).

As described above, the microchip 71 is arranged on the display unit 15 of the tablet terminal device 73, and the optical analysis is performed in the above-described manner. The observation target light from the specimen is detected by the built-in camera 75, and the information obtained upon the detection is operated and calculated by the control unit 72 of the tablet terminal device 73. The analysis result obtained upon the operation and the calculation is displayed in the display unit 15.

The analyzing apparatus of the above-described embodiment uses the portable tablet terminal device 73 that has the operating and calculating function, as the analyzing unit. The analyzing apparatus of the above-described embodiment is configured to use the microreactor, which includes the microchip 71 placed on the display unit 15, to perform the isolation, separation, synthesis, extraction, analysis and the like for a trace amount of specimen.

Because the table terminal device 73 that has the electric power feeding port is used, the light emitted from the external light source, which receives the electricity from the electric power feeding port, can be used as the light for the detection of the analysis target and as the energy for the driving (e.g., for driving the air pumps). In addition, the operating and calculating unit in the tablet terminal device 73 can be used to operate, calculate and analyze the detection data obtained from the analysis target. It is also possible to display the analysis results on the display unit 15.

The external light source 3 may include the LEDs or LDs. By appropriately selecting the intensity and/or the spectral characteristics of the light emitted from each of the LEDs (or LDs) of the external light source 3, it is possible to use the light, which has a particular wavelength suitable for the measurement of the reaction of the specimen introduced to the fluid passage of the microchip 71, at the light intensity suitable for the measurement. The external light source 3 can be integrated into the external light source module 9, which has the electric power feeding terminal electrically connected to the external light source 3. Thus, as the electric power feeding terminal is coupled to the electric power feeding port of the tablet terminal device 73, the external light source module 9 becomes integral with the tablet terminal device 73.

In the analyzing apparatus of this embodiment, therefore, the external light source module 9 and the operating and calculating unit are integrated in the portable tablet terminal device 73. The analyzing apparatus of this embodiment can perform the analysis with the tablet terminal device 73 and the microchip 71 that does not include active components. Unlike the conventional analyzing apparatus, the analyzing apparatus of this embodiment does not need a dedicated control component (see FIGS. 22A-22C). Thus, the analyzing apparatus of this embodiment is small in size and possible to carry. The analyzing apparatus of the above-described embodiment can conduct the testing (measurement and analysis) in a short time at a location where the analysis should be carried out, and can also provide the precise evaluation and analysis results. Consequently, the analyzing apparatus of this embodiment meets the demand for, for example, the POCT (point of care testing) in the life science technology.

The analyzing system itself is included in the tablet terminal device 73. Thus, the microchip 71 does not include the analyzing system. Then, the microchip 71 can be an inexpensive and ordinary microchip which does not include active components. Such microchip can be used as a disposal component. Accordingly, it is not necessary to use an expensive microchip that is integrated with a detection system having, for example, organic EL elements integrated therein.

Because the light from the external light source 3 is introduced to the microchip 71 by the optical fibers 69, the optical analysis is not affected even if the position of the microchip 71 on the tablet terminal device 73 changes to a certain extent. Thus, the position adjustment of the microchip 71 on the tablet terminal device 73 is not necessary before starting the measurement and during the measurement, and the measurement can be performed quickly.

As described above, the analyzing apparatus of this embodiment uses the communication function of the tablet terminal device 73, if necessary, to download the software for the analysis, depending upon the details of the analysis to be performed on the measurement target. Thus, the analyzing apparatus of this embodiment can be employed as an analyzing apparatus that can cope with a variety of analyses for a variety of specimens.

While a dedicated detection system is prepared for a particular analysis in the conventional technology, the single analyzing apparatus of this embodiment can deal with various types of analyses. Thus, it is not necessary to prepare many analyzing apparatuses that are customized for the respective analyses.

The analyzing apparatus of this embodiment can easily perform the logging of the measured data into the tablet terminal device 73. Thus, the analyzing apparatus of this embodiment does not need a dedicated storage unit. Also, it is easy to build up the analyzing system that uses the communication function. The displaying manner of the display unit 15 may be customized depending upon the selection of emitted light color and the indication (displaying) of the analysis data.

It should be noted that the processing device used in the present invention is not limited to the tablet terminal device (e.g., tablet computer). Any suitable processing device may be employed in the analyzing apparatus as long as the processing device has a display unit (or display portion) and possesses an operating and calculating function. For example, a personal computer, a portable telephone, a smartphone may be employed as the processing device.

### Fourth Embodiment

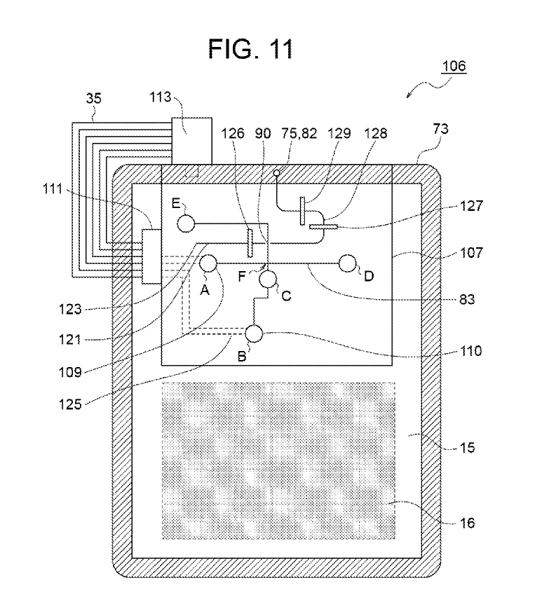

Referring now to FIG. 11, the fourth embodiment of the present invention will be described. FIG. 11 shows a schematic configuration of the optical processing apparatus 106. In this embodiment, the external light source such as the one shown in FIG. 1B is attached to the microchip 107. The processing unit such as a tablet terminal device 73 or the like feeds the electric power to the external light source. The external light source is detachable from the microchip 107.

The analyzing apparatus of this embodiment is similar to the analyzing apparatus of the first embodiment except for the following points: (1) the analyzing apparatus of this embodiment has the external light source that is detachable from the microchip 107, as described above. (2) Piezoelectric pumps (piezoelectric diaphragm pumps) 109 and 110 are provided at the ports A and B of the microchip 107 instead of the light-driven air pumps. (3) The optical analyzing unit to carry out the optical analysis in the fluid passage FE has a different structure than the first embodiment.

In the following description, therefore, the external light source, the piezoelectric pumps and the optical analyzing unit will only be described. For the sake of easier understanding, the size of the microchip 107 is depicted in an exaggerated scale in FIG. 11. Thus, the actual size relationship between the tablet terminal device 73 and the microchip 107 is different from that illustrated in FIG. 11.

### The External Light Source

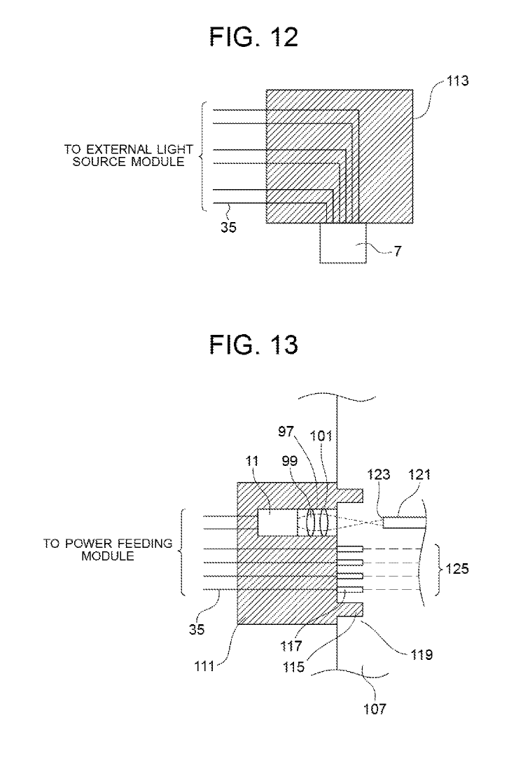

FIG. 12 shows an exemplary configuration of the electric power feeding module 113 to feed electricity to the external light source module 111, which includes the external light source, to be attached to the microchip 107 (will be described later). The electric power feeding module 113 shown in FIG. 12 has an electric power feeding terminal (e.g., USB terminal 7). When the USB terminal 7 is engaged with (inserted into) the USB port (power feeding port) of the tablet terminal device 73 (processing device), the tablet terminal device 73 can feed electric power to the electric power feeding module 113. Power feeding lines 35 extend to the external light source module 111 from the power feeding module 113 to feed the electric power to the external light source module 111.

FIG. 13 shows an exemplary configuration of the external light source module 111.

The external light source module 111 of FIG. 13 is attached to, for example, the side face of the microchip 107. That face of the external light source module 111 which is attached to the microchip 107 has a plurality of projections such as protruding portions 115 for fixing, and pins 117 for power feeding. The side face of the microchip 107 to which the external light source module 111 is attached has a plurality of cavities (depressions) 119 that correspond to the projections such as the protruding portions 115 and the power feeding pins 117. As the protruding portions 115 and the power feeding pins 117 are received in the cavities 119, the external light source module 111 is attached to (detachably fixed to) the microchip 107.

The external light source module 111 shown in FIG. 13 has a single light source therein. The light source may include an LED 11. The LED 11 emits light at an optimal wavelength with a sufficient light intensity as light to be directed to the fluid, which contains the specimen introduced to the fluid passage of the microchip 107.

The external light source module 111 has a condensing hole 97 on the light emitting side of the LED 11. In the condensing hole 97, there are provided a collimator lens 99 and a condensing lens 101 in this order from the LED 11 side.

The light emitted from the LED 11 is incident to the collimator lens 99 and collimated, and then the collimated light is incident to the condensing lens 101. The light incident to the condensing lens 101 proceeds in the microchip 107 and is condensed to a light introducing end (light incident face) 123 of the light guiding path 121 (will be described later).

The positions of the protruding portions 115 and the pins 117 and the positions of the receiving holes 119 are decided such that the light emitted from the external light source (LED) 11 is incident to the light introducing end 123 of the optical path 121.

As the external light source module 111 is attached to the microchip 107, the power feeding pins 117 protruding from the external light source module 111 are coupled to the pump power feeding lines 125 extending from the piezoelectric pumps 109 and 110 disposed at the ports A and B of the microchip 107 (will be described later).

### The Piezoelectric Pumps

The piezoelectric pumps 109 and 110 disposed at the ports A and B are diaphragm pumps which are actuated by piezoelectric elements. The microchip 107 has the pump power feeding lines 125 to feed electricity to the piezoelectric pumps 109 and 110. As described above, when the external light source module 111 is attached to the microchip 107, the pump power feeding lines 125 are electrically coupled to the power feeding pins 117 of the external light source module 111.

### The Optical Analyzing Unit

As shown in FIG. 11, the optical analyzing unit to carry out the optical analysis in the fluid passage FE includes a filter 126, the light introducing path 121, a filter 127, the light introducing path 128, a filter 129 and a light introducing hole 82 for the built-in camera.

As described above, the light emitted from the LED 11 of the external light source module 111 attached to the microchip 107 is incident to the collimator lens 99 of the external light source module 111 for collimation, the collimated light is incident to the condensing lens 101, and the condensed light is incident to the microchip 107 from the side face of the microchip 107. The light incident to the microchip 107 proceeds in the microchip 107 is condensed to the light introducing end 123, which is the light incident face of the light introducing path 121 (will be described later).

The light introducing path 121 guides the light emitted from the external light source module 111. The light emitting portion of the light introducing path 121 opens at the lateral portion 90 of the fluid passage FE. The light introducing path 121 is made from a material such as silicone resin. The material of the light introducing path 121 may be decided such that the refractive index of the material is greater than the material (silicone resin) of the microchip 107 and the refractive index of the air, and such that the light emitted from the display unit 15 can pass through the light introducing path 121 (the light introducing path 121 permeates the light from the display unit 15).

The light introducing end 123 (i.e., the light incident face) of the light introducing path 121 is located at a position to receive the light from the external light source module 111. The light exit face of the light introducing path 121 is located at a position to face the lateral portion 90 of the fluid passage FE that is filled with the solution of the specimen diluted by the buffer solution (e.g., PBS).

The light emitted from the LED 11 and incident to the light introducing path 121 from the light introducing end 123 is diffused light (light that diffuses after condensation (concentration)). Therefore, part of the light is incident to the upper and lower walls (top and bottom walls) and the right and left walls of the light introducing path 121 before the light reaches the exit (light emitting end face) of the light introducing path 121. Because the refractive index of the light introducing path 121 is greater than the refractive index of the microchip 107 and greater than the refractive index of the air, that part of the light which is incident to the upper, lower, right and left walls of the light introducing path 121 is totally reflected and proceeds toward the exit of the light introducing path 121 if the incident angle of that part of the light which is incident to the upper, lower, right and left walls of the light introducing path 121 is equal to or greater than the critical angle of the light introducing path 121.

Accordingly, the light introducing path 121 serves as a waveguide that guides the light, which is emitted from the display unit, to the solution of the specimen in the fluid passage FE such that the specimen is irradiated with the light.

A filter 126 is disposed in the light introducing path 121 between the light inlet 123 and a point arriving at the lateral portion 90 of the fluid passage FE. The filter 126 cuts off that wavelength component which does not contribute to the excitation of the specimen, from the light introduced from the light inlet 123 of the light introducing path 121.

Thus, the light incident to the light introducing path 121 is introduced to the lateral portion 90 of the fluid passage FE via the filter 126, and the solution of the specimen diluted by the buffer solution (PBS) in the fluid passage FE is excited by the light.

The excited specimen emits light (e.g., fluorescence) depending upon the physical property of the specimen. The emitted light is used as the observation target light. The emitted light is guided by the light guiding path 128 such that the light passes through the filters 127 and 129 and the light introducing hole 82 in this order and is incident to the built-in camera 75 of the tablet terminal device 73. Then, the light is detected by the built-in camera 75.

Filters 127 and 129 are disposed in the light guiding path 128 between the lateral portion 90 of the fluid passage FE and the light introducing hole 82 for the built-in camera 75.

The light incident to the light incident face of the light guiding path 128 may include not only the observation target light (e.g., fluorescence) emitted from the specimen but also other light. Part of the light emitted from the LED 11, which does not contribute to the excitation of the specimen, may proceed across the fluid passage FE and be incident to the light incident face (inlet) of the light guiding path 128. Such light becomes a noise to the optical analysis of the specimen, and should be removed.

The filters 127 and 129 cut off the excitation light. For example, each of the filters 127 and 129 may include a dielectric optical element (notch filter) or a color glass filter (absorption filter).

The light introducing hole 82 for the built-in camera in this embodiment is the same as the light introducing hole 82 of the third embodiment so that the detail of the light introducing hole 82 is omitted.

As shown in FIG. 9, the camera light introducing hole 82 has the inclined surface 82a that is inclined relative to the surface of the microchip 107. By appropriately deciding (setting) the angle of the inclined surface 82a in consideration of the critical angle of the microchip 107 and other factors, the observation target light introduced from the light guiding path 128 is turned downward by the inclined surface 82a (toward the built-in camera 75) and condensed.

### Fifth Embodiment

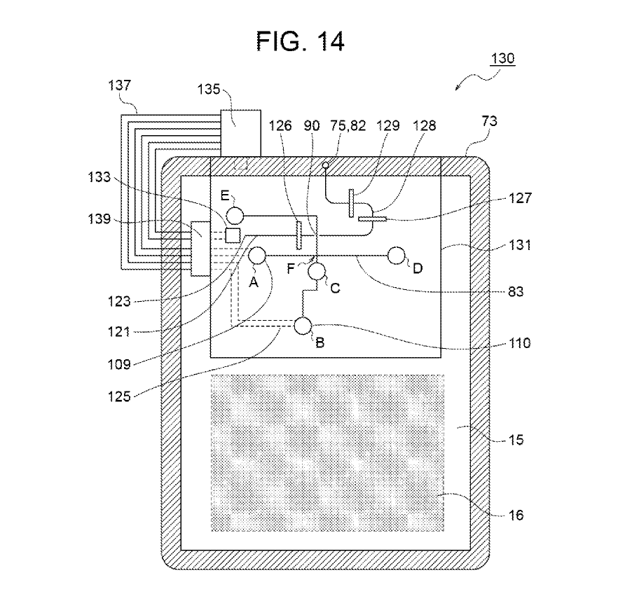

Referring to FIG. 14, a fifth embodiment of the present invention will be described. FIG. 14 illustrates an exemplary configuration of the optical processing apparatus 130. In this embodiment, the external light source (e.g., the one shown in FIG. 2C) is built in the microchip itself, and the processing device such as the tablet terminal device feeds the electric power to the external light source.

The optical analyzing apparatus of this embodiment employs the piezoelectric pumps and the optical analyzing unit of the fourth embodiment. The optical analyzing apparatus of this embodiment is similar to the optical analyzing apparatus of the fourth embodiment except for the external light source being built in the microchip.

Thus, the following description only deals with the external light source. It should be noted that for the sake of easier understanding, the size of the microchip 131 is exaggerated in FIG. 14. The actual size relationship between the tablet terminal device 73 and the microchip 131 is different from the size relationship depicted in FIG. 14.

### The External Light Source

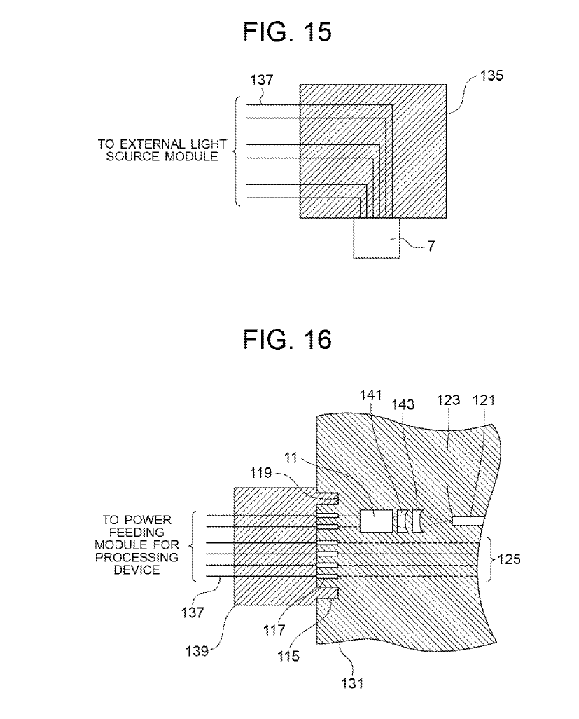

FIG. 15 shows the configuration of a power feeding module 135 on the processing device side. The power feeding module 135 is one of the power feeding modules adapted to feed the electric power to the external light source which is built in the microchip 131. The power feeding module 135 on the processing device side may be referred to as “first power feeding module 135.” The first power feeding module 135 shown in FIG. 15 has a power feeding terminal (e.g., USB terminal 7). When the USB terminal 7 is engaged in (inserted in) the USB port (power feeding port) of the tablet terminal device 73 (processing device), the tablet terminal device 73 can feed electricity to the first power feeding module 135. The power feeding module 135 has power feeding lines 137 to feed the electricity to the external light source.

FIG. 16 shows an exemplary configuration of a power feeding module 139 on the microchip side, and the external light source that is built in the microchip 131.

The power feeding module 139 on the microchip side may be referred to as “second power feeding module 139.” The second power feeding module 139 shown in FIG. 16 is attached to, for example, the lateral face of the microchip 131. That face of the second power feeding module 139 which is attached to the microchip 131 has a plurality of projections such as protruding portions 115 for fixing, and pins 117 for power feeding. The lateral face of the microchip 131 to which the second power feeding module 139 is attached has a plurality of cavities (depressions) 119 that correspond to the projections such as the protruding portions 115 and the power feeding pins 117. As the protruding portions 115 and the power feeding pins 117 are received in the cavities 119, the second power feeding module 139 is attached to (detachably fixed to) the microchip 131.

In FIG. 16, the single external light source is built in the microchip 131. The external light source is, for example, an LED 11. The LED 11 emits light that has an optimal wavelength with a suitable light intensity as the light to be directed to the fluid that contains the specimen introduced to the fluid passage of the microchip 131.

On the light emitting side of the LED 11 in the microchip 131, there are provided a collimator lens 141 and a condensing lens 143 in this order.

The light emitted from the LED 11 is incident to the collimator lens 141 and collimated. The collimated light is incident to the condensing lens 143. The light incident to the condensing lens 143 proceeds in the microchip 131, and is condensed to the light incident face (light inlet or end face) 123 of the light introducing path 121. This is similar to the fourth embodiment shown in FIG. 13.

The positions of the projections (protruding portions 115 and pins 117) of the second power feeding module 139 and the receiving holes 119 of the microchip 131 are decided such that when the second power feeding module 139 is attached to the microchip 131, some of the power feeding pins 117 protruding from the second power feeding module 139 are connected to the power feeding lines of the LED 11 (external light source 11), and the remainder of the power feeding pins 117 is connected to the power feeding lines 125 that extend to the piezoelectric pumps 109 and 110 disposed at the ports A and B of the microchip 131 (see FIG. 14).

As described above, the electricity feeding to the external light source 11 and the piezoelectric pumps 109 and 110 is carried out by a power feeding unit that includes the first power feeding module 135, the power feeding lines 137, and the second power feeding module 139. The first power feeding module 135 is electrically connectable to the processing device when the first power feeding module 135 is attached to the processing device. Thus, when the second power feeding module 139 is attached to the microchip 131, the processing device is electrically connectable to the microchip 131. The control unit of the processing device controls the electricity feeding to the external light source 11 and the piezoelectric pumps 109 and 110 of the microchip 131 from the processing device.

The external light source 11 and the power feeding module 139 on the microchip side (second power feeding module) in the fifth embodiment correspond to the external light source module in the fourth embodiment.

Similar to the fourth embodiment, the piezoelectric pumps 109 and 110 are disposed at the ports A and B shown in FIG. 14. The piezoelectric pumps 109 and 110 are diaphragm pumps and actuated by the piezoelectric elements. As illustrated in FIG. 14, the optical analyzing unit to be used to perform the optical analysis in the fluid passage FE includes the filter 126, the light introducing path 121, the light guiding path 128, the filters 127 and 129, and the camera light introducing hole 82.

Similar to the fourth embodiment, the light emitted from the external light source 11 is incident to the light introducing end (light inlet) 123 of the light introducing path 121 via the collimator lens 141 and the condensing lens 143, and is emitted from the light exit of the light introducing path 121 located at a position facing the lateral portion 90 of the fluid passage FE. The fluid passage FE is filled with the solution of the specimen which is diluted by the buffer solution such as PBS. Because the filter 126 is provided in the light introducing path 121 between the light inlet 123 and the light exit, located at the lateral portion 90 of the fluid passage FE, to cut off the wavelength component other than the wavelength component to be used for the excitation of the specimen, the solution of the specimen which is diluted by the buffer solution (PBS) in the fluid passage FE is excited by the light emitted from the light exit of the light introducing path 121.

The fluorescence from the excited specimen is the observation target light, and is guided by the observation light guiding path 128 such that the observation target light is incident to the built-in camera 75 of the tablet terminal device 73 via the filters 127 and 129 and the camera light introducing hole 82. Thus, the observation target light is detected by the built-in camera 75.

The filters 127 and 129 (notch filters or absorption filters) are provided in the observation target light guiding path 128 between the lateral portion 90 of the fluid passage FE and the camera light introducing hole 82 to eliminate a noise light (noise to the light (fluorescence) emitted from the specimen).

### Sixth Embodiment

### Modification to the Fifth Embodiment

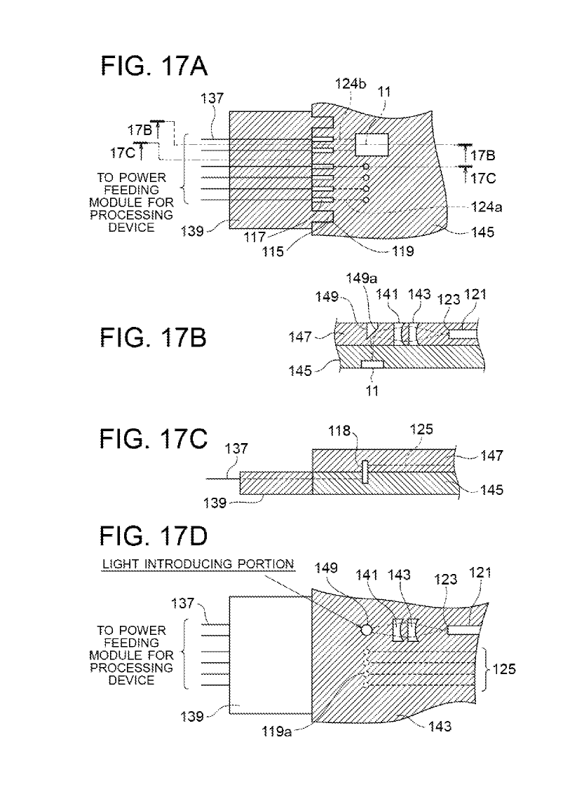

In the analyzing apparatus of the fifth embodiment, the optical analyzing unit and the liquid conveying unit such as the piezoelectric pumps 109 and 110 are incorporated in the microchip 131. The present invention is not limited to such configuration. As shown in FIGS. 17A to 17D, for example, the microchip 131 may be divided into a specimen light measuring chip 147 and an external light source chip 145. The light analyzing unit and the liquid conveying unit (e.g., piezoelectric pumps 109 and 110) may be incorporated in the specimen light measuring chip 147. The external light source may be built in the chip 145. The chips 145 and 147 may be laminated.

When such configuration is employed, the second power feeding module 139 (power feeding module on the microchip side) shown in FIG. 16 is attached to, for example, the lateral face of the external light source chip 145 as shown in FIG. 17A. That face of the second power feeding module 139 which is attached to the external light source chip 145 has a plurality of projections such as protruding portions 115 for fixing, and pins 117 for power feeding. The lateral face of the external light source chip 145 to which the second power feeding module 139 is attached has a plurality of cavities (depressions) 119 that correspond to the projections such as the protruding portions 115 and the power feeding pins 117. As the protruding portions 115 and the power feeding pins 117 are received in the cavities 119, the second power feeding module 139 is attached (detachably fixed to) the external light source chip 145.

FIG. 17A shows an example of the external light source chip 145 that has a single built-in external light source. The light source may include an LED 11. The LED 11 emits light at an optimal wavelength with a sufficient light intensity as light to be directed to the fluid, which contains the specimen introduced to the fluid passage of the microchip 131.

FIG. 17B is a cross-sectional view taken along the line 17B-17B in FIG. 17A. As shown in FIG. 17B, the LED 11 emits light upward in FIG. 17B. This light proceeds through the external light source chip 145 and the specimen light measuring chip 147, and is directed to the light introducing hole 149 (light inlet) for the optical measurement of the specimen.

As shown in FIG. 17B, the light introducing hole 149 is formed in that face (upper face) of the specimen light measuring chip 147 which is opposite the face (lower face) that contacts the external light source chip 145. The light introducing hole 149 has a bottom wall 149a which is inclined at a predetermined angle relative to the upper face of the specimen light measuring chip 147.

The specimen light measuring chip 147 is made from silicone resin such as PDMS. In general, the refractive index of the silicone resin is greater than the refractive index of the air (atmosphere). Thus, when the light emitted from the external light source chip 145 is incident to the inclined bottom wall 149a of the light introducing hole 149 at an incident angle that is equal to or greater than a critical angle of the specimen light measuring chip 147 (silicone resin) to the air, then the light is totally reflected by the bottom wall 149a. By appropriately deciding the angle of the inclined wall 149a, the light emitted in the upward direction in FIG. 17 from the external light source chip 145 is reflected (turned) to a transverse direction (lateral direction) by the inclined wall 149a of the light introducing hole 149.

As shown in FIG. 17B, the inclined wall 149a of the light introducing hole 149 is formed to turn the light such that the turned light proceeds to the collimator lens 141 disposed in the specimen light measuring chip 147. On the light exit side of the collimator lens 141 in the specimen light measuring chip 147, there are provided the condensing lens 143 and the light guiding path 121 in this order. The light emitted from the LED 11 built in the external light source chip 145 is turned by the inclined wall 149a of the light introducing hole 149 and incident to the collimator lens 141 for collimation. Then, the collimated light is incident to the condensing lens 143. The light incident to the condensing lens 143 proceeds in the microchip and is condensed to the light incident face (light inlet) 123 of the light guiding path 121.

FIG. 17C is a cross-sectional view taken along the line 17C-17C in FIG. 17A. FIG. 17D is a top view of the specimen light measuring chip 147. As understood from FIGS. 17C and 17D, the external light source chip 145 has a plurality of power feeding pins 118 that protrude toward the specimen light measuring chip 147. The specimen light measuring chip 147 has a plurality of cavities (depressions) 119a that correspond to the power feeding pins 118 of the external light source chip 145.

As the second power feeding module (power feeding module on the microchip side) 139 is attached to the external light source chip 145, the power feeding pins 118 of the external light source chip 145 are electrically connected to some of the power feeding pins 117 extending from the second power feeding module 139 by the power feeding lines 124a. The remainder of the power feeding pins 117 extending from the second power feeding module 139 is electrically connected to the power feeding lines 124b of the LED 11 (i.e., external light source).

In the sixth embodiment, therefore, the external light source that is built in the external light source chip 145, and the second power feeding module 139 constitute in combination the external light source module.

When the specimen light measuring chip 147 is arranged on the external light source chip 145, the power feeding pins 118 protruding from the external light source chip 145 toward the specimen light measuring chip 147 are received in the depressions 119a of the specimen light measuring chip 147, and electrically connected to the pump power feeding lines 125 provided in the specimen light measuring chip 147.

The power feeding lines 125 for the pumps are coupled to the piezoelectric pumps 109 and 110 disposed at the ports A and B (see FIG. 14). This is similar to the fourth embodiment.

### Seventh Embodiment

The analyzing apparatuses in the above-described embodiments use the optical analyzing apparatus developed by the inventors (e.g., the analyzing apparatus disclosed in Japanese Patent Application No. 2013-35581) but relies upon the light emitted from the external light source, rather than the light emitted from the display portion of the tablet terminal device, when the microchip is irradiated with the light.

For example, the external light source may include one or a plurality of LEDs or LDs. By appropriately selecting the light intensity and spectral characteristic of the light emitted from each of the LEDs or LDs (external light sources), it is possible to use the light having a particular wavelength and a light intensity suitable for observation of the reaction of the specimen introduced to the fluid passage of the microchip. Also, by appropriately selecting the characteristic of the light emitted from each of the LEDs or LDs (external light sources), e.g., by selecting the pulsed light or the coherent light in consideration of given conditions, it is possible to use the light that is suitable for the observation of the reaction of the specimen introduced to the fluid passage of the microchip.

The external light source may be used not only as a light source to emit the light instead of the light emitted from the display unit (display portion) of the tablet terminal device, but also as a light source to emit light that is used together with the light emitted from the display unit of the tablet terminal device. For example, when the specimen-containing fluid introduced to the microchip is irradiated with the light (visible light) emitted from the display unit to cause the specimen-containing fluid to absorb the light and emit light, the specimen-containing fluid may be irradiated with another light (second light) such as ultraviolet light. This changes the absorption of the visible light, which is emitted from the display unit, by the specimen-containing fluid.

Thus, the external light source may be used as a (second) light source to emit the above-mentioned “second light.”

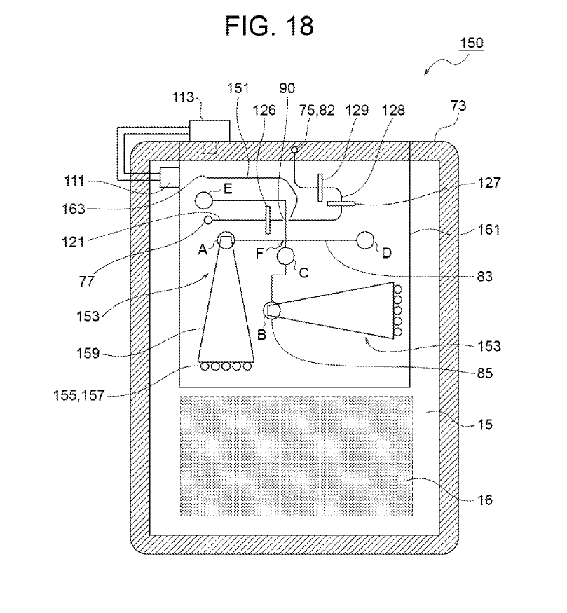

FIG. 18 shows a schematic configuration of an optical processing apparatus 150. The optical processing apparatus 150 uses the fourth embodiment (FIG. 11), which is derived from the optical analyzing apparatus developed by the inventors (Japanese Patent Application No. 2013-35581), together with a second light source and a second light introducing path 151. The second light source is the external light source (power feeding module 113 and external light source module 111). The second light introducing path 151 guides the light emitted from the external light source. Two light capturing units (mechanism) 153 are provided.

The light capturing units 153 for capturing the light emitted from the display unit 15 are described in Japanese Patent Application No. 2013-35581, and the detail of the light capturing units 153 is not described here. The light emitted from the display unit 15 is captured (collected) by the light capturing units 153, and is guided to the ports A and B where in the light-driven air pumps (micropumps) 85 are disposed. Each of the light capturing units 153 includes a liquid crystal light collimator microlens array (collimator lens array 155), an optical path changing holes 157 for turning the light, emitted from the collimator lens array 155, into the microchip, and a flat plane tapered light guiding path 159. The optical path changing holes 157 change the optical path of the light emitted from the collimator lens array 155 such that the light proceeds in the microchip.

The light emitted from the display unit 15 is incident to the microchip 161 from the light introducing hole 77, and is guided by the light introducing path 121 such that the solution that contains the specimen introduced to the microchip 161 is irradiated with this light.

The light emitted from the display unit 15 is used to actuate the light-driven air pumps 85, and fluid that contains the specimen introduced to the microchip 161 is irradiated with this light. Then, the light emitted from the specimen-containing fluid is detected. This is described in Japanese Patent Application No. 2013-35581, the entire disclosure thereof is incorporated herein by reference.

The external light source shown in FIG. 18 has a similar configuration to the external light source of the fourth embodiment shown in FIG. 11. The external light source is detachably fixed to (engaged with) the microchip 161, and the processing device (e.g., tablet terminal device 73) feeds the electric power to the external light source.

The structure of the power feeding module 113 for feeding electricity to the external light source module 111 is similar to the one shown in FIG. 12. The power feeding module 113 has a power feeding terminal (e.g., USB terminal). As the USB terminal is engaged in the USB port (power feeding port) of the processing device (table terminal device) 73, the tablet terminal device 73 can feed the electric power to the power feeding module 113. The power feeding module 113 has power feeding lines to feed the electric power to the external light source module 111. Unlike the fourth embodiment, the power feeding module 113 does not feed the electric power to the piezoelectric pumps. Therefore, the electric feeding lines to the piezoelectric pumps are not seen in FIG. 18.

The structure of the external light source module 111 is similar to the external light source module shown in FIG. 13. For example, the external light source module 111 is attached to the lateral face of the microchip 161. That face of the external light source module 111 which is attached to the microchip 161 has a plurality of projections such as protruding portions for fixing, and pins for power feeding. The lateral face of the microchip 161 to which the external light source module 111 is attached has a plurality of cavities (depressions) that correspond to the projections such as the protruding portions and the power feeding pins. When the projections of the external light source module 111 are received in the corresponding depressions of the microchip 161, the external light source module 111 is attached (detachably fixed to) the microchip 161.

The external light source module 111 may include a single light source therein. For example, the light source may include an LED that emits an ultraviolet ray.

As shown in FIG. 13, the condensing hole is formed on the light emitting side of the LED. In the condensing hole, there are provided a collimator lens and a condensing lens in this order when viewed from the LED.

Light emitted from the LED is incident to the collimator lens for collimation. The collimated light is then incident to the condensing lens. The light incident to the condensing lens proceeds in the microchip, and is condensed to the light incident face (light inlet or light introducing end) 163 of the second light introducing path 151 shown in FIG. 18.

The positions of the projections (protruding portions and the power feeding pins) of the external light source module 111 and the depressions of the microchip 161 are decided such that the light emitted from the external light source (LED) is incident to light incident face 163 of the second light introducing path 151.

The second light introducing path 151 guides the light (“second light”) emitted from the external light source module 111. The light exit of the second light introducing path 151 is located (opens) in the vicinity of the lateral portion 90 of the fluid passage FE. The lateral portion 90 of the fluid passage FE is the portion which is also irradiated with the light (“first light”) emitted from the display unit 15. The second light introducing path 151 is made from a material such as silicone resin. The material of the second light introducing path 151 is selected (decided) such that the refractive index of the second light introducing path 151 is greater than the refractive index of the material of the microchip (e.g., silicone resin) and the refractive index of the air, such that the light emitted from the display unit 15 can pass through the second light introducing path 151 (the light introducing path 151 permeates the light from the display unit 15).

The light emitted from the external light source (LED) and incident to the second light introducing path 151 from the second light introducing end 163 is diffused light (light that diffuses after condensation (concentration)). Therefore, part of the light is incident to the upper and lower walls (top and bottom walls) and the right and left walls of the second light introducing path 151 before the light reaches the exit (light emitting end face) of the second light introducing path 151. Because the refractive index of the second light introducing path 151 is greater than the refractive index of the microchip 161 and greater than the refractive index of the air, that part of the light which is incident to the upper, lower, right and left walls of the second light introducing path 151 is totally reflected and proceeds toward the exit of the light second introducing path 151 if the incident angle of that part of the light which is incident to the upper, lower, right and left walls of the second light introducing path 151 is equal to or greater than the critical angle of the second light introducing path 151.

Thus, the second light introducing path 151 serves as a waveguide to guide the light, which is emitted from the external light source, to the solution of the specimen in the fluid passage FE such that the specimen is irradiated with this light.

In the seventh embodiment, therefore, when the optical analysis is performed with the light emitted from the display unit 15, the fluid that contains the specimen introduced to the microchip 161 is irradiated with the second light from the external light source.

### Eighth Embodiment

The tablet terminal device 73 that is used in the analyzing apparatus of the above-described third, fourth, fifth, sixth and seventh embodiments has the camera 75 which is built in the tablet terminal device 73. The built-in camera 75 detects the measurement target light, and the control unit 72 that is also built in the tablet terminal device 73 operates and calculates the detected data and information.

On the other hand, the tablet terminal device 165 used in the analyzing apparatus of the eighth embodiment does not have a built-in camera. The observation target light from the specimen is observed by human eyes.

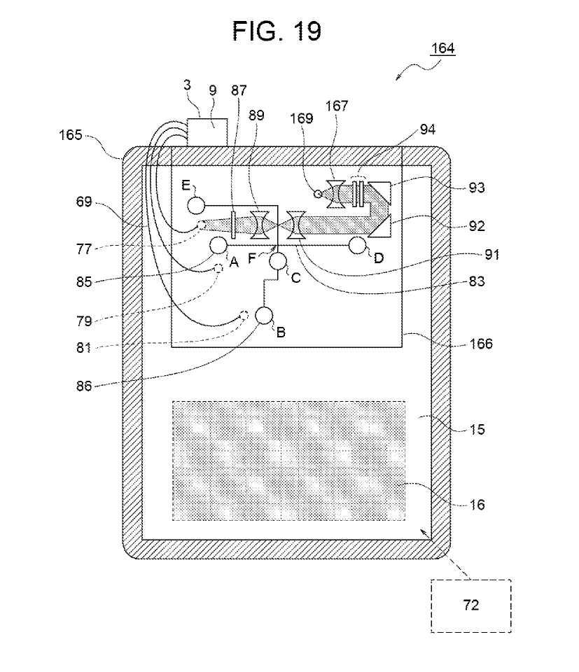

FIG. 19 schematically illustrates an optical processing apparatus 164. The optical analyzing apparatus of FIG. 19 is similar to the optical analyzing apparatus of the third embodiment shown in FIG. 5. The optical analyzing apparatus of FIG. 19 is different from the optical analyzing apparatus of the third embodiment in that the optical analyzing apparatus of FIG. 19 does not have the built-in camera, the camera light introducing hole, and the condensing hole, but it has a third lens 167 and an observation hole 169.

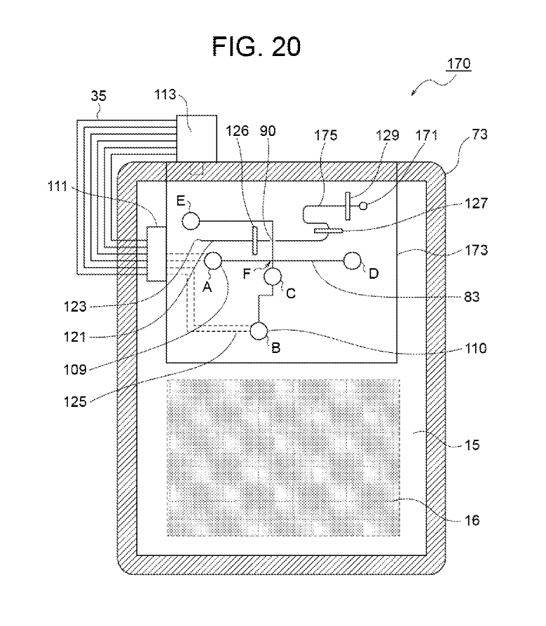

FIG. 20 schematically illustrates an optical processing apparatus 170. The optical analyzing apparatus of FIG. 20 is similar to the optical analyzing apparatus of the fourth embodiment shown in FIG. 11. The optical analyzing apparatus of FIG. 20 is different from the optical analyzing apparatus of the fourth embodiment in that the optical analyzing apparatus of FIG. 20 does not have the built-in camera and the camera light introducing hole, but it has an observation hole 171.

In FIG. 19, the light analyzing unit used to carry out the optical analysis on the light in the fluid passage FE includes the radiated light introducing hole 77, the first lens 89, the second lens 91, the two parallel light filters (first filter 92 and second filter 93), the filter 94, the third lens 167 and the observation hole 169.

The structures and roles of the first lens 89, second lens 91, two parallel light filters 92 and 93, and filter 94 are the same as those in the first embodiment. Thus, the details of the first lens 89, second lens 91, two parallel light filters 92 and 93, and filter 94 are not described here.

The third lens 167 is defined by a hollow space formed in the microchip 166. The light incident surface of the third lens 167 is concave, and the light exiting surface of the third lens 167 is also concave. The concave shape of the light incident surface and the concave shape of the light exiting surface are decides such that the light passing through the third lens 167 is incident to the observation hole 169 and condensed in the observation hole 169.

A dye (pigment, colorant) may be embedded in the observation hole 169 such that the dye may emit light when the dye is irradiated with the observation target light having a predetermined wavelength. Thus, the analysis result can be confirmed (recognized) by visual inspection (by human eyes).

In FIG. 20, the optical analyzing unit to carry out the optical analysis in the fluid passage FE includes a light introducing hole 123, the filter 126, the light introducing path 121, the observation target light guiding path 175, the filters 127 and 129, and the observation hole 171. The structures and roles of the light introducing hole 123, the filter 126, the light introducing path 121, the observation target light guiding path 175, and the filters 127 and 129 are the same as those in the second embodiment. Thus, the details of the light introducing hole 123, the filter 126, the light introducing path 121, the observation target light guiding path 175, and the filters 127 and 129 will not be described here.

The observation target light is introduced to the observation hole 171 from the light guiding path 175. As described above, the dye (pigment, colorant) is embedded in the observation hole 171 such that the dye may emit light when, for example, the dye is irradiated with the observation target light having a predetermined wavelength. Thus, the analysis result of the specimen can be confirmed (recognized) by visual inspection (by human eyes).

Accordingly, the analyzing apparatus of the fifth embodiment may be used in the optical analysis with the analysis result being recognized by human eyes. Because the analyzing apparatus of the fifth embodiment does not need a built-in camera, the analyzing apparatus of the fifth embodiment can be manufactured with less cost than the analyzing apparatuses of the third and fourth embodiments.

Although the single microchip possesses various functions in the third, fourth and fifth embodiments, the present invention is not limited to such configuration. For example, a microchip having an optical analyzing unit may be prepared, another microchip having one or more light-driven air pumps may be prepared, and other microchip(s) may be prepared. Then, these microchips may be laminated or arranged next to each other to configure a set of microchips that serves in combination as the single microchip of the third, fourth or fifth embodiment.

### Ninth Embodiment

### Modifications to the Third, Fourth, Fifth, Sixth, Seventh and Eighth Embodiments

The microchip of any of the third to eighth embodiments may include a pretreatment module that has a filtering function and/or other function(s). The pretreatment module may be integrated in the microchip. The pretreatment module may include a filter for separation (e.g., for separating a blood cell or blood plasma) that has a pillar structure. Another pretreatment module may include a filter for capturing and separating a solid matter or particle. Such filters are disclosed, for example, in Patent Literatures 3, 4 and 5. The pretreatment module may separate substances which are not the specimen (e.g., blood cells, blood plasma, undesired solid matters, and undesired particles), from the specimen-containing fluid, so that the substances which are not necessary to be irradiated with the light are excluded from the specimen-containing fluid.

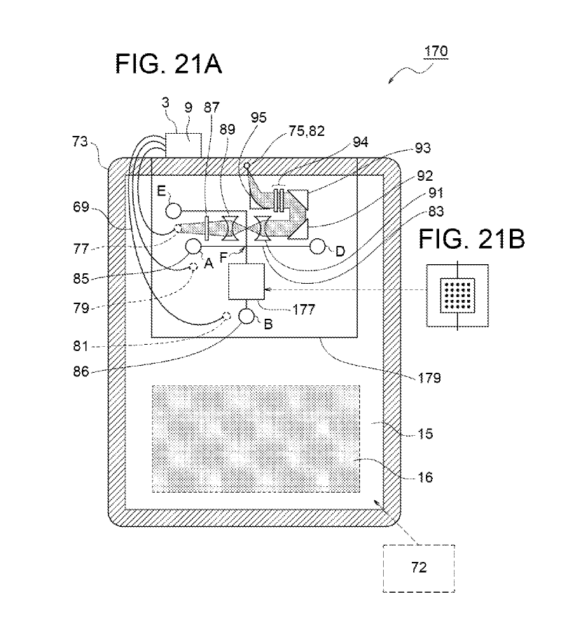

Referring to FIGS. 21A and 21B, the pretreatment filter 177 incorporated in the microchip shown in FIG. 5 will be described. FIG. 21A illustrates a schematic configuration of the optical processing apparatus 176 that has the pretreatment filter 177 between the port B and the intersection F to the micro fluid passage 83. The pretreatment filter 177 has a pillar structure as shown in FIG. 21B, and is configured to separate the blood cells and/or blood plasma. The pretreatment filter 177 can perform the pretreatment such as separating the blood cells, blood plasma and the like from the liquid sent to the intersection F form the port B in the fluid passage.

FIG. 21A shows the modification to the embodiment of FIG. 3. Specifically, the analyzing apparatus of the third embodiment additionally includes the pretreatment filter 177 in FIG. 21A. It should be noted that the fourth embodiment or the fifth embodiment may also include the pretreatment filter 177.

In FIG. 21A, the pretreatment filter 177 is incorporated in the microchip 179. Alternatively, a chip that has the same function as the pretreatment filter 177 may be prepared beforehand, and this chip may be laminated on the microchip of the third embodiment (or any other suitable embodiment) with a fluid passage being connected to impart the pretreatment function to the microchip, or this chip may be arranged next to the microchip with a fluid passage being connected to impart the pretreatment function to the microchip.

While certain embodiments have been described in the foregoing, these embodiments have been presented by way of example only, and are not intended to limit the scope of the present invention. The novel apparatuses (devices) and methods thereof described herein may be embodied in a variety of other forms. Furthermore, various omissions, substitutions and changes in the form of the apparatuses (devices) and methods thereof described herein may be made without departing from the gist of the present invention. The accompanying claims and their equivalents are intended to cover such forms or modifications as would fall within the scope and gist of the present invention.

The present application is based upon and claims the benefit of a priority from Japanese Patent Application No. 2013-32245, filed Feb. 21, 2014, and the entire contents of which are incorporated herein by reference.

What is claimed is:

1. A method for optical analysis comprising:
preparing a portable terminal device having an operating and calculating unit, a light receiving unit, and a display unit configured to display processing results of the operating and calculating unit;
preparing a microchip having a light inlet portion and a light outlet portion, but having no light source, the microchip being configured to hold a specimen in a first optical path extending from the light inlet portion to the light outlet portion;
preparing a second optical path configured to guide light exiting from the light outlet portion of the microchip to the light receiving unit;
introducing light into the light inlet portion of the microchip to irradiate the specimen in the first optical path with the light;
guiding the light, which is emitted from the irradiated specimen, to the light receiving unit through the second optical path; and
analyzing the light, which is received at the light receiving unit, by the operating and calculating unit.

2. The method according to claim 1, wherein said introducing light into the light inlet portion of the microchip includes using an external light source other than the portable terminal device to introduce the light into the light inlet portion.

3. The method according to claim 2, wherein said introducing light into the light inlet portion of the microchip includes causing the portable terminal device to feed electricity to the external light source.

4. The method according to claim 3, wherein the portable terminal device further includes a control unit configured to control the display unit, and during said introducing light into the light inlet portion of the microchip, the control unit controls the display unit to reduce or stop light emission from the display unit.

5. The method according to claim 4 further including, prior to said introducing light into the light inlet portion of the microchip, determining whether or not a remaining battery energy of the portable terminal device is equal to or greater than a value which is sufficient to feed the electricity to the external light source.

6. The method according to claim 2, wherein light emitted from the external light source is different from light emitted from the display unit in terms of at least one of wavelength and intensity.

7. The method according to claim 2, wherein the light emitted from the external light source includes pulsed light, coherent light, terahertz light, and/or polarized light.

8. The method according to claim 1 further including, prior to said introducing light into the light inlet portion of the microchip, determining whether the light receiving unit functions normally.

9. A system for optical analysis comprising:
a portable terminal device having an operating and calculating unit, a light receiving unit, and a display unit configured to display processing results of the operating and calculating unit;
a microchip having a light inlet portion and a light outlet portion such that a specimen is held in a first optical path extending from the light inlet portion to the light outlet portion, the specimen being irradiated with light for analysis of the specimen; and
a second optical path configured to guide light exiting from the light outlet portion of the microchip to the light receiving unit such that the operating and calculating unit of the portable terminal device analyzes the light which is received at the light receiving unit.

10. The system according to claim 9 further including an external light source configured to receive electricity from the portable terminal device, and to emit the light with which the specimen is irradiated.

11. The system according to claim 9, wherein the second optical path has a light condensing portion configured to enhance an intensity of the light directed to the light receiving unit.

12. The system according to claim 9, wherein the portable terminal device further includes a control unit configured to control the display unit in order to reduce or stop light emission from the display unit.

13. The system according to claim 10 further including a unit for determining whether or not a remaining battery energy of the portable terminal device is equal to or greater than a value which is sufficient to feed the electricity to the external light source.

14. The system according to claim 10, wherein light emitted from the external light source is different from light emitted from the display unit in terms of at least one of wavelength and intensity.

15. The system according to claim 10, wherein the light emitted from the external light source includes pulsed light, coherent light, terahertz light, and/or polarized light.

16. The system according to claim 9 further including a unit for determining, prior to introducing the light into the light inlet portion of the microchip, whether the light receiving unit functions normally.

17. A program that causes the portable terminal device to perform the method according to claim 1.
